(19) United States
(12) Patent Application Publication
(10) Pub. No.: US 2019/0053788 A1
(43) Pub. Date: Feb. 21, 2019

(54) METHOD AND ULTRASOUND APPARATUS FOR PROVIDING ANNOTATION RELATED INFORMATION

(71) Applicant: Samsung Electronics Co., Ltd., Gyeonggi-do (KR)

(72) Inventors: Jong-chae MOON, Seoul (KR); Seo-lynn PARK, Seoul (KR); Eun-mee SHIN, Gyeonggi-do (KR)

(21) Appl. No.: 16/048,450

(22) Filed: Jul. 30, 2018

(30) Foreign Application Priority Data
Aug. 17, 2017 (KR) 10-2017-0104141

(51) Int. Cl. A61B 8/00; A61B 8/08

(52) U.S. Cl. CPC A61B 8/468; A61B 8/13; A61B 8/465; A61B 8/5246

(57) ABSTRACT

Provided is a method of providing annotation-related information, the method including displaying, on a display of a ultrasound apparatus, an ultrasound image obtained by using a probe; receiving an input user requesting an annotation setup interface; identifying a diagnostic field corresponding to the ultrasound image; determining text items, based on the identified diagnostic field; and displaying, on a touchscreen of the ultrasound apparatus, the annotation setup interface including the text items.

### CROSS-REFERENCE TO RELATED APPLICATION

This application is based on and claims priority under 35 U.S.C. § 119 to Korean Patent Application No. 10-2017-0104141, filed on Aug. 17, 2017, in the Korean Intellectual Property Office, the disclosure of which is incorporated by reference herein in its entirety.

### BACKGROUND

### 1. Field

The disclosure relates to a method and ultrasound apparatus for providing annotation-related information by providing an interface configured to set an annotation via a touchscreen.

### 2. Description of Related Art

Ultrasound apparatuses transmit an ultrasound signal from a body surface of an object toward a predetermined part in the body, and obtain a sectional image of soft tissue or an image of blood flow by using information of the ultrasound signal reflected from tissue in the body.

Ultrasound apparatuses are small and relatively inexpensive, and display images in real time. Also, because ultrasound apparatuses are safe due to the lack of radioactive exposure from X-rays or the like, they are widely used together with other image diagnosis apparatuses such as X-ray diagnosis apparatuses, computerized tomography (CT) scanners, magnetic resonance imaging (MRI) apparatuses, nuclear medical diagnosis apparatuses, or the like.

The above information is presented as background information only to assist with an understanding of the present disclosure. No determination has been made, and no assertion is made, as to whether any of the above might be applicable as prior art with regard to the present disclosure.

### SUMMARY

Provided is a method of providing an annotation setup interface, which can be based on a diagnostic department, the method being performed by an ultrasound apparatus to allow a user, such as an user (e.g., a medical expert) to conveniently set an annotation when the user manipulates a probe with one hand and a control panel with the other hand.

Provided is a method of providing an annotation setup interface that is useful to a user, such as an user, the method being performed by an ultrasound apparatus that selectively further uses, in addition to information about a diagnostic department, at least one of information about an examinee, information about the user, measurement result information, probe information, information about a function used when an ultrasound image is obtained, and location information in which the ultrasound apparatus is installed.

Provided is a method of providing annotation-related information, the method being performed by an ultrasound apparatus adaptively determining items, an arrangement, a layout, or the like in an annotation setup interface so as to allow an user to conveniently set an annotation.

Additional aspects will be set forth in part in the description which follows and, in part, will be apparent from the description, or may be learned by practice of the presented embodiments.

In accordance with an aspect of the disclosure, a method, performed by an ultrasound apparatus, of providing annotation-related information, includes displaying, on a display of the ultrasound apparatus, an ultrasound image obtained by using a probe; receiving an input requesting an annotation setup interface; identifying a group corresponding to the ultrasound image; determining text items, based on the identified group; and displaying, on a touchscreen of the ultrasound apparatus, the annotation setup interface comprising the text items.

user.

In accordance with another aspect of the disclosure, an ultrasound apparatus includes a display configured to display an ultrasound image obtained by using a probe; an input interface configured to receive an input requesting an annotation setup interface; and a processor configured to determine text items, based on a group corresponding to the ultrasound image, and display the annotation setup interface comprising the text items on a touchscreen forming a portion of the input interfaceuser.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram for describing an ultrasound system, according to an embodiment;

FIG. 2 is a flowchart for describing a method, performed by an ultrasound apparatus, of providing annotation-related information, according to an embodiment;

FIG. 3 is a diagram for describing an annotation setup interface, according to an embodiment;

FIG. 4 is a diagram for describing an annotation setup interface that is provided according to a diagnostic department, according to an embodiment;

FIG. 5 is a diagram for describing a method of determining a layout of an annotation setup interface, according to an embodiment;

FIG. 6 is a diagram for describing an operation of optimizing a layout of an annotation setup interface, the operation being performed by the ultrasound apparatus, according to an embodiment;

FIG. 7 is a diagram for describing an operation, performed by the ultrasound apparatus, of optimizing a layout of an annotation setup interface by using at least one of text item selecting pattern information and incorrect-input pattern information, according to an embodiment;

FIG. 8 is a diagram for describing an operation, performed by the ultrasound apparatus, of adjusting a size of a text included in a text item or adjusting a color of the text item, according to an embodiment;

FIG. 9 is a flowchart for describing a method, performed by the ultrasound apparatus, of adjusting a layout of an annotation setup interface by using examinee-related information, according to an embodiment;

FIG. 10 is a diagram for describing an operation, performed by the ultrasound apparatus, of providing an annotation setup interface by using an examination history of an examinee, according to an embodiment;

FIG. 11 is a diagram for describing an operation, performed by the ultrasound apparatus, of providing an annotation setup interface by using body characteristic information of an examinee, according to an embodiment;

FIG. 12 is a flowchart for describing a method, performed by the ultrasound apparatus, of adjusting a layout of an annotation setup interface by using user-related information, according to an embodiment;

FIGS. 13A and 13B are diagrams for describing an operation, performed by the ultrasound apparatus, of determining a layout of an annotation setup interface according to by which hand an user manipulates a control panel, according to an embodiment;

FIG. 14 is a flowchart for describing a method, performed by the ultrasound apparatus, of providing an annotation setup interface by using measurement result information, according to an embodiment;

FIG. 15 is a diagram for describing an operation, performed by the ultrasound apparatus, of determining text items by using measurement result information, according to an embodiment;

FIG. 16 is a diagram for describing an operation, performed by the ultrasound apparatus, of providing an annotation setup interface by using information about a hospital in which the ultrasound apparatus is installed, according to an embodiment;

FIG. 17 is a flowchart for describing a method, performed by the ultrasound apparatus, of generating an annotation, based on an input, according to an embodiment;

FIG. 18 is a diagram for describing an operation, performed by the ultrasound apparatus, of providing an image for guiding a method of setting an annotation, according to an embodiment;

FIG. 19 is a diagram for describing an operation, performed by the ultrasound apparatus, of generating an annotation, based on an input received via an annotation setup interface, according to an embodiment;

FIG. 20 is a diagram for describing an operation, performed by the ultrasound apparatus, of amending an annotation, based on an input received via an annotation setup interface, according to an embodiment;

FIG. 21 is a flowchart for describing a method, performed by the ultrasound apparatus, of moving a position of an annotation, in response to a touch input, according to an embodiment;

FIG. 22 is a diagram for describing an operation, performed by the ultrasound apparatus, of receiving an input of moving a position of an annotation via a touchscreen, according to an embodiment;

FIG. 23 is a diagram for describing an operation, performed by the ultrasound apparatus, of receiving an input of moving a position of an annotation by using a trackball in a control panel, according to an embodiment; and

FIGS. 24 and 25 are block diagrams for describing configurations of the ultrasound apparatus, according to embodiments.

### DETAILED DESCRIPTION

All terms including descriptive or technical terms which are used herein should be construed as having meanings that are obvious to one of ordinary skill in the art. However, the terms may have different meanings according to an intention of one of ordinary skill in the art, precedent cases, or the appearance of new technologies. Also, some terms may be arbitrarily selected by the applicant, and in this case, the meaning of the selected terms will be described in detail in the detailed description of the disclosure. Thus, the terms used herein have to be defined based on the meaning of the terms together with the description throughout the specification.

Throughout the specification, when a part “includes” or “comprises” an element, unless there is a particular description contrary thereto, the part can further include other elements, not excluding the other elements. In the following description, terms such as “unit” and “module” indicate a unit for processing at least one function or operation, wherein the unit and the module may be embodied as hardware or embodied by combining hardware and software.

Throughout the specification, an “ultrasound image” refers to an image of an object obtained by using an ultrasound signal. In the present specification, an “object” may include an examinee (a human or an animal), or a part of an examinee (a human or an animal). For example, the object may include, but is not limited to, an organ such as the liver, the heart, the kidney, the womb, the brain, a breast, the abdomen, the thyroid, and the prostate, or a blood vessel. In the present specification, an examinee may be expressed as a patient.

The ultrasound images may be realized in a variety of modes. For example, the ultrasound image may be, but is not limited to, one of a brightness (B) mode image, a color (C) mode image, a Doppler (D) mode image, a motion (M) mode image, an elastic mode image, and an elastic image, wherein the B mode image shows as brightness, a magnitude of an ultrasound echo signal reflected from an object, the C mode image shows, as color, a velocity of a moving object by using the Doppler effect, the D mode image shows, in a spectrum form, an image of a moving object by using the Doppler effect, the M mode image shows motions of an object according to time at a constant position, the elastic mode image shows an image of a difference between responses when compression is or is not applied to an object, and the elastic image is an image of a shear wave. In addition, according to an embodiment, the ultrasound image may be a two-dimensional (2D) image, a three-dimensional (3D) image, or a four-dimensional (4D) image.

useruserThroughout the specification, an “annotation” may refer to at least one word that simply expresses characteristic information related to an ultrasound image, and may include information about an object from which the ultrasound image is obtained, a measurement result, characteristics about an examinee, or the like.

The present disclosure will now be described more fully with reference to the accompanying drawings for one of ordinary skill in the art to be able to perform the present disclosure without any difficulty. The present disclosure may, however, be embodied in many different forms and should not be construed as being limited to the embodiments set forth herein. In addition, portions irrelevant to the description of the present disclosure will be omitted in the drawings for a clear description of the present disclosure, and like reference numerals will denote like elements throughout the specification.

As used herein, the term “and/or” includes any and all combinations of one or more of the associated listed items. Expressions such as “at least one of,” when preceding a list of elements, use the entire list of elements and do not use the individual elements of the list.

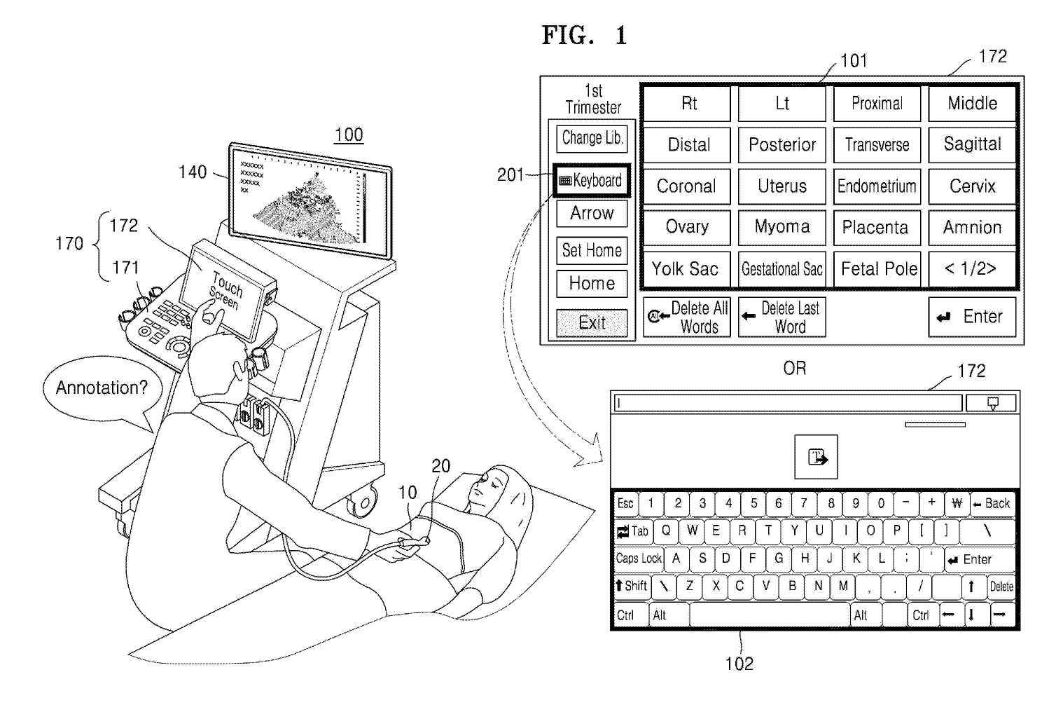

FIG. 1 is a diagram for describing an ultrasound system, according to an embodiment.

FIG. 1 illustrates an ultrasound user examining an examinee 10 with an ultrasound probe 20. While the user examines the patient, the user may use either control panel 171 or touchscreen 172 to annotate or label the ultrasound image 140. The ultrasound image 140 can be maintained by keeping the probe 20 at the same position of the examinee 10. Certain embodiments facilitate annotation of the ultrasound image 140 using the user's other hand.

Referring to FIG. 1, an ultrasound apparatus 100 may include a display 140, an input interface 170, a probe 20, and an interface for connection with the probe 20 (hereinafter, a probe connector). In this regard, the input interface 170 may include a control panel 171 including hardware buttons, and a touchscreen 172. Hereinafter, each element of the ultrasound apparatus 100 will now be described in detail.

According to the present embodiment, the display 140 may be a main screen configured to display an ultrasound image or information of an examinee 10. An user may recognize a condition of the examinee 10 via the ultrasound image displayed on the display 140. For example, the user may detect a lesion on the examinee 10 or may check the health of a fetus, by using the ultrasound image displayed on the display 140. The display 140 may include, but is not limited to, at least one of liquid crystal display, a thin film transistor-liquid crystal display, an organic light-emitting diode display, a flexible display, and a 3D display. The display 140 may include a touch pad (a touch capacitive type touch pad, a pressure resistive type touch pad, an infrared beam sensing type touch pad, a surface acoustic wave type touch pad, an integral strain gauge type touch pad, a piezo effect type touch pad, or the like).

The input interface 170 may be a unit through which the user inputs data to control the ultrasound apparatus 100. To facilitate annotation of ultrasound images, the input interface 170 includes a graphical user interface that allows fast entry of many common fields of data that an ultrasound user is likely to use annotate the ultrasound image.

The input interface 170 may include the control panel 171 including hardware buttons to control functions provided by the ultrasound apparatus 100, and the touchscreen 172 configured to display a graphical user interface (GUI), a menu list, or the like.

According to the present embodiment, the control panel 171 may include, but is not limited to, the hardware buttons such as a trackball, a knob button, a probe button, a power button, a scan button, a patient button, an ultrasound image selection button, or the like. The patient button may refer to a button to select a patient to receive an ultrasound diagnosis. The probe button may refer to a button to select a probe to be used in the ultrasound diagnosis. The scan button may refer to a button to rapidly compensate for an ultrasound image by using a preset parameter value. A store button may refer to a button to store an ultrasound image. The ultrasound image selection button may refer to a button to pause a real-time displayed ultrasound image so as to make one still ultrasound image displayed on a screen.

The touchscreen 172 may be configured to detect not only a touch input position, a touched area but also detect a touch input pressure. Also, the touchscreen 172 may be configured to detect both a real touch and a proximate touch. The real touch indicates a case in which a pointer is actually touched on a screen, and the proximate touch indicates a case in which the pointer is not actually touched on the screen but is adjacent to the screen by a preset distance. In this regard, the pointer refers to a touching tool for touching or proximately touching a particular portion of a displayed screen image. Examples of the pointer may include an electronic pen, a finger, or the like. For convenience of description, hereinafter, it is assumed that the pointer is a finger.

The touchscreen 172 may detect a touch gesture of a user (e.g., the user). Examples of the touch gesture of the user which are described in the present specification may include a tap gesture, a touch & hold gesture, a double-tap gesture, a drag gesture, a panning gesture, a flick gesture, a drag and drop gesture, a swipe gesture, a pinch gesture, or the like.

The touchscreen 172 may display a plurality of control items. The plurality of control items refer to user-selectable items that include, but are not limited to, a menu, a control button, a mode selection button, a shortcut icon, a control interface, a function key, a setting window (e.g., an annotation setup interface), or the like.

According to the present embodiment, each of the plurality of control items may be associated with at least one function. For example, the plurality of control items may include, but are not limited to, a 2D button, a 3D button, a 4D button, a color button, a PW button, an M button, a SonoView button (a button to check a pre-stored image), a more button, a measure button, an annotation button, a biopsy button (a button to guide a position to which a needle is to be inserted), a depth button, a focus button, a gain button, a frequency button, or the like.

According to the present embodiment, the hardware buttons included in the control panel 171 may be implemented as software and then may be displayed on the touchscreen 172. For example, a Freeze button to display a still image may be arranged as a hardware button on the control panel 171 and may be displayed as a software button on the touchscreen 172. Here, a software button may be a user interface (UI) object that is implemented as software and displayed on a screen image. For example, the software button may be an icon, a setting key, a menu, or the like which are displayed on the touchscreen 172. A function matched with each software button may be executed, in response to a touch input of touching each software button.

According to the present embodiment, in a case where the input interface 170 is formed as the touchscreen 172, the control panel 171 and the touchscreen 172 may be the same.

The probe 20 may transmit an ultrasound signal to the examinee 10, in response to a driving signal from an ultrasound transmitting and receiving unit, and may receive an echo signal reflected from the examinee 10. The probe 20 includes a plurality of transducers that vibrate in response to a received electric signal and generate an ultrasound wave that is acoustic energy. In the present specification, a type of the probe 20 may vary. For example, the probe 20 may include, but is not limited to, a convex probe, a linear array probe, a sector probe, and a phased array probe.

According to the present embodiment, the user may manipulate the input interface 170 in one hand while the user captures an ultrasound image by holding the probe 20 in the other hand. Users may prefer to manipulate the probe 20 using their preferred hand (the hand they write with). Certain embodiments can facilitate annotation of the ultrasound image 140 using the less favored hand. For example, the user may hold the probe 20 in a right hand and may select, by using a left hand, the annotation button included in the input interface 170. In this case, the ultrasound apparatus 100 may provide an annotation setup interface 101 via the touchscreen 172. The annotation setup interface 101 may include common words (e.g., Rt, Lt, Proximal, Middle, Distal, Posterior, or the like) which are frequently used as annotations. However, when many common words are simply listed on the annotation setup interface 101, it is difficult for the user to find an appropriate word from among the common words. Here, the user may select a keyboard button 201 so as to directly input an annotation. When the user selects the keyboard button 201, the annotation setup interface 101 may display a keyboard 102. However, because the user holds the probe 20 in the right hand, it may be difficult to manipulate the keyboard 102 and input texts by using only the left land.

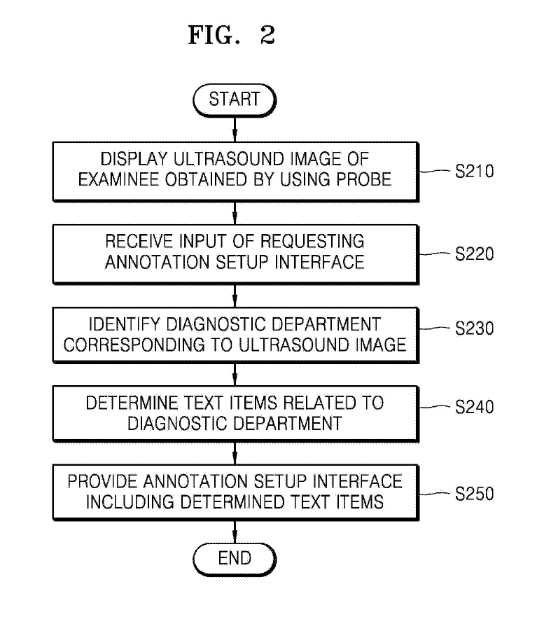

Thus, hereinafter, a method of providing an annotation setup interface, the method being provided to allow an user to conveniently set an annotation by using one hand, will now be described in detail with reference to FIG. 2.

FIG. 2 is a flowchart for describing a method, performed by the ultrasound apparatus 100, of providing annotation-related information, according to an embodiment.

In operation S210, the ultrasound apparatus 100 may display, on the display 140, an ultrasound image of an examinee which is obtained by using the probe 20.

The ultrasound image may be variously displayed. For example, the ultrasound image may be, but is not limited to, at least one of a brightness mode (B mode) image, a color mode (C mode image), a Doppler mode (D mode) image, a motion mode (M mode) image, and an elastic mode (E mode) image. In addition, the ultrasound image may be a 2D image, a 3D image, or a 4D image.

According to the present embodiment, the ultrasound apparatus 100 may display the ultrasound image of the examinee on both the display 140 and the touchscreen 172.

The ultrasound apparatus 100 may receive an input related to the ultrasound image, the input including include a variety of information, such as diagnostic department information (e.g., the departments of urology, obstetrics and gynecology, orthopedic surgery, cardiovascular, endocrinology, general diagnostic center, pediatrics, thorax and cardiology, radiation, neurosurgery, etc.), user's characteristic information (e.g., a position of an user, a posture of the user, whether the user is left-handed, etc.), examinee's personal information (gender, age, etc.), examinee's characteristic information (e.g., absence of a right kidney, etc.), examinee's diagnosis history information, diagnosis target part information (e.g., a breast, an abdomen, a musculoskeletal system, blood vessels, a thyroid, etc.), examinee's posture information (the examinee lies supine or on his/her stomach, the examinee lies supine with his/her head turned to the right or left, the examinee lies with his/her head back, the examinee is sitting up, or the like), probe selection information (e.g., identification information about a selected probe, etc.), or the like. In addition, the ultrasound apparatus 100 may display, on the display 140, the ultrasound image and at least one of examinee-related information, user-related information, identification information about a selected probe, and measurement result information.

According to the present embodiment, the ultrasound image may be, but is not limited to, a real-time captured image, an image fetched from among ultrasound images stored in a storage, or an ultrasound image received from an external server.

In operation S220, the ultrasound apparatus 100 may receive an input from the user who requests an annotation setup interface. For example, the user may select an annotation button to set an annotation related to the ultrasound image displayed on the display 140.

The ultrasound apparatus 100 may receive an input of selecting the annotation button included in the control panel 171. Alternatively, the ultrasound apparatus 100 may receive an input of touching an annotation setting icon (e.g., T/annotation) displayed on the touchscreen 172.

In operation S230, the ultrasound apparatus 100 may identify a diagnostic field that corresponds to the ultrasound image. The diagnostic field may be expressed as an application.

The ultrasound apparatus 100 may identify information about the diagnostic field that is input by the user or is preset. For example, in a case where a gynecology department uses the ultrasound apparatus 100, an input diagnostic field (or an application) may be a gynecology or breast. Also, in a case where a urology department uses the ultrasound apparatus 100, even if the user does not separately input urology as the diagnostic field, the diagnostic field (or the application) may be set to the urology as a default.

In operation S240, the ultrasound apparatus 100 may determine text items based on the diagnostic field.

According to the present embodiment, the ultrasound apparatus 100 may determine the text items corresponding to the diagnostic department, by using a table where common words that are mainly used in each diagnostic field are defined. For example, in a case where the diagnostic field is the breast examination field, the ultrasound apparatus 100 may determine likely text that would be used in an ultrasound in the breast examination field. For example, the text items, ‘Rt (right) and Lt (left)’ respectively indicating right and left breasts, ‘Upper, Lower, Medial, and Lateral’ indicating sectional directions, ‘Nipple, Axillary, Lymph Node, and Nodule’ indicating parts, or the like, may be used.

According to the present embodiment, the ultrasound apparatus 100 may determine the text items that are likely to be used during an ultrasound breast examination by using measurement result information related to the ultrasound image. For example, the ultrasound apparatus 100 may determine words indicating information related to a size or a position of a lesion, as the text items to be included in the annotation setup interface.

According to the present embodiment, the ultrasound apparatus 100 may determine the text items, based on a diagnosis target part or a type of the probe 20 connected to the ultrasound apparatus 100, in addition to the diagnostic field. For example, in a case where the diagnostic field is gynecology, and the probe 20 is a linear probe used in a breast ultrasound diagnosis, the ultrasound apparatus 100 may determine words that are likely to be used and are related to a breast ultrasound, as the text items. Also, in a case where the diagnostic field is the gynecology field, and the probe 20 is a convex probe, the ultrasound apparatus 100 may determine words that are likely to be used and related to a womb ultrasound, as the text items

According to the present embodiment, the ultrasound apparatus 100 may determine the text items, based on functions used in a process of obtaining the ultrasound image, in addition to the diagnostic field. For example, when an elastic ultrasound image of the examinee is obtained by using an elastic mode function, the ultrasound apparatus 100 may determine, as the text items, words that are likely to be used and related to a grade of elasticity (an elasticity coefficient), and words related to a lesion detected from the elastic ultrasound image.

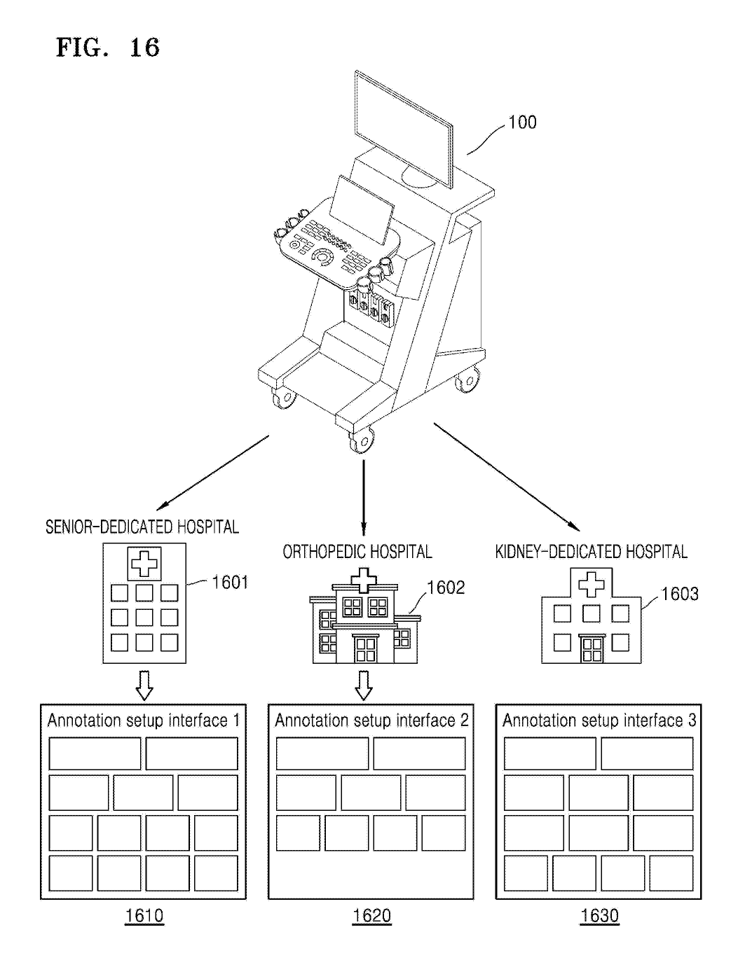

According to the present embodiment, the ultrasound apparatus 100 may determine the text items, based on information about a known diagnostic field that the hospital in which the ultrasound apparatus 100 is installed is most likely to conduct. For example, in a case where the hospital in which the ultrasound apparatus 100 is installed is an urology-specialized clinic, the ultrasound apparatus 100 may determine, as the text items, words that are likely to be used and related to the urology field, the words including hydrocele, varicocele, fluid, hernia, sperm cord, etc. An operation, performed by the ultrasound apparatus 100, of providing the annotation setup interface according to a type of a hospital having the ultrasound apparatus 100 will be described in detail below with reference to FIG. 16.

In operation S250, the ultrasound apparatus 100 may provide, to the touchscreen 172, the annotation setup interface including the determined text items.

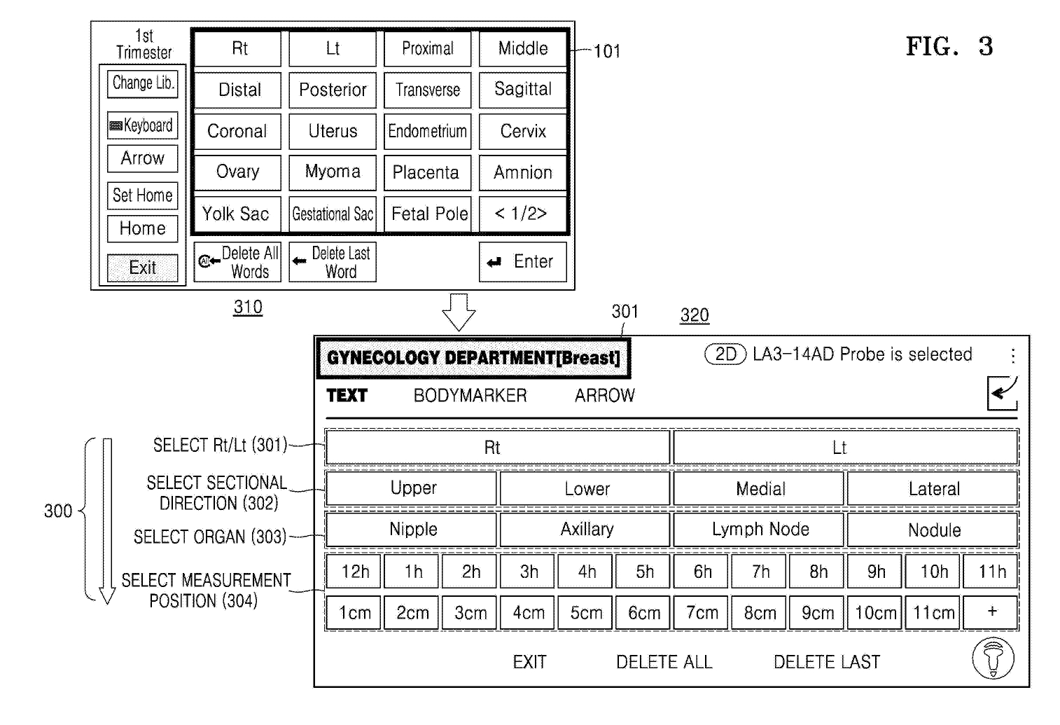

According to the present embodiment, the determined text items may be arranged on the annotation setup interface, according to semantic correlations. For example, the ultrasound apparatus 100 may group the text items according to the semantic correlations. Then, the ultrasound apparatus 100 may arrange the text items on rows, according to the groups, respectively. An operation, performed by the ultrasound apparatus 100, of grouping the text items according to the semantic correlations will be described in detail below with reference to FIG. 3.

According to the present embodiment, a layout of the annotation setup interface may vary. For example, positions of the text items included in the annotation setup interface, sizes of the text items, colors of the text items, fonts of texts included in the text items, sizes of the texts, and colors of the texts may be variously realized.

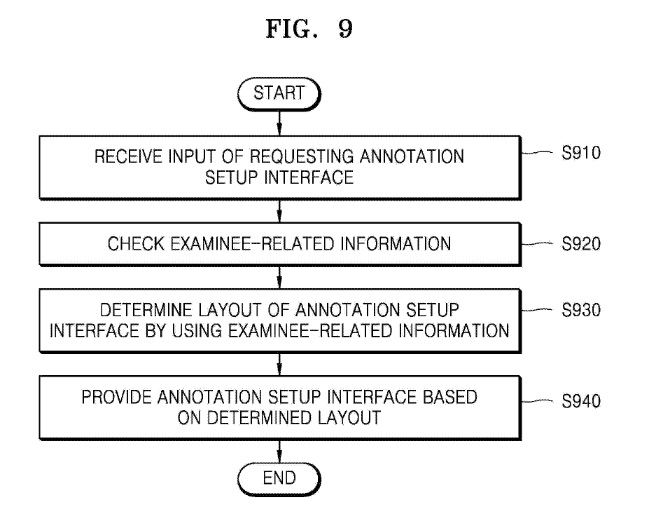

According to the present embodiment, the ultrasound apparatus 100 may determine a layout of the annotation setup interface by using the examinee-related information. The examinee-related information may include, but is not limited to, at least one of an examination history of the examinee, an examination posture of the examinee, and physiological characteristic information of the examinee. An operation, performed by the ultrasound apparatus 100, of determining the layout of the annotation setup interface by using the examinee-related information will be described in detail below with reference to FIG. 9.

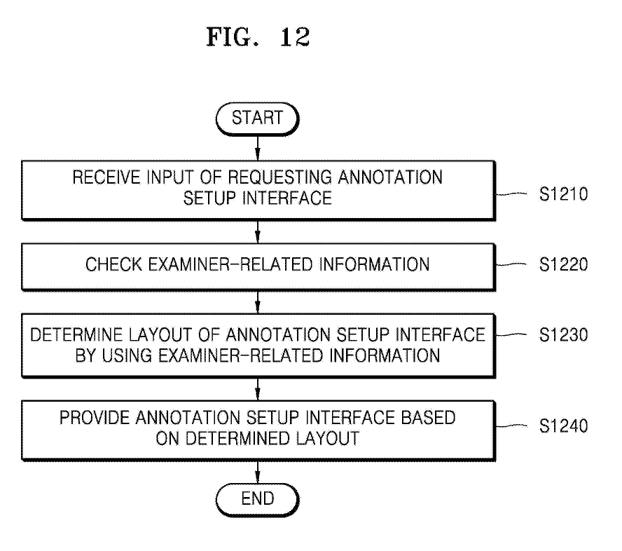

According to the present embodiment, the ultrasound apparatus 100 may determine the layout of the annotation setup interface by using the user-related information about an user. In this regard, the user-related information may include, but is not limited to, information about with which hand the user uses the probe 20 (e.g., a left hand or a right hand), information about a pattern by which the user selects text items, information about a position of the user, information about a posture of the user, or the like. An operation, performed by the ultrasound apparatus 100, of determining the layout of the annotation setup interface by using the user-related information will be described in detail below with reference to FIG. 12.

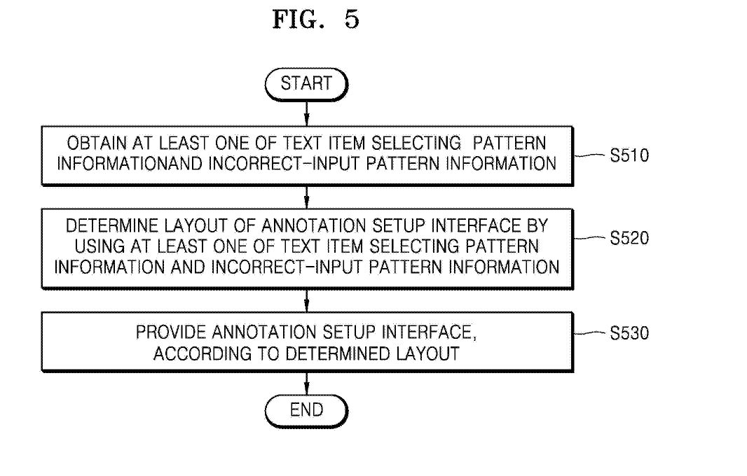

According to the present embodiment, the ultrasound apparatus 100 may determine, by using at least one of text item selecting pattern information and incorrect-input pattern information, at least one of positions of the text items included in the annotation setup interface, sizes of the text items, colors of the text items, fonts of texts included in the text items, sizes of the texts, and colors of the texts. An operation, performed by the ultrasound apparatus 100, of determining the layout of the annotation setup interface by using at least one of the text item selecting pattern information and the incorrect-input pattern information will be described in detail below with reference to FIG. 5.

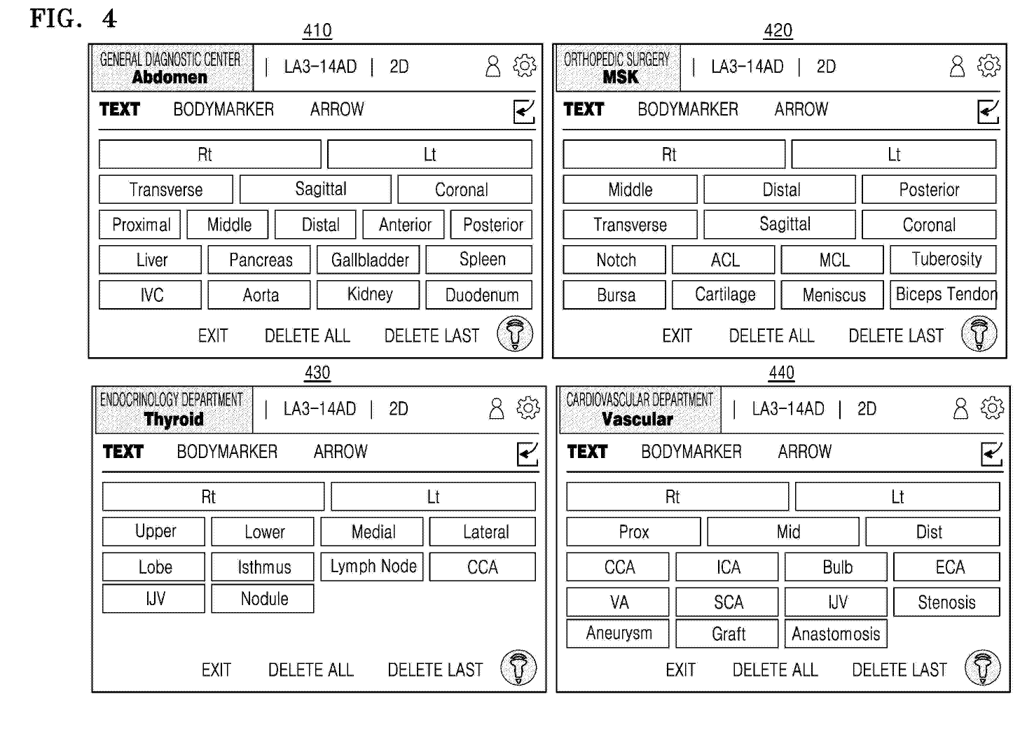

According to the present embodiment, the ultrasound apparatus 100 may provide the annotation setup interface including the text items corresponding to the diagnostic department, in response to an input of requesting the annotation setup interface from the user, such that the ultrasound apparatus 100 may allow the user to rapidly find appropriate text items configuring an annotation. Hereinafter, with reference to FIGS. 3 and 4, the annotation setup interface based on diagnostic departments will now be described in detail.

FIG. 3 is a diagram for describing an annotation setup interface, according to an embodiment. With reference to FIG. 3, an example in which an user scans a breast ultrasound image will now be described.

Referring to an execution screen 310 illustrated in FIG. 3, when the ultrasound apparatus 100 receives an input requesting an annotation setup interface from the user, the ultrasound apparatus 100 may provide a general annotation setup interface 101 regardless of a diagnostic field (e.g., a breast examination field). In this regard, on a first page of the general annotation setup interface 101, text items (e.g., nipple, axillary, lymph node, nodule) related to breast examinations are not displayed. Thus, the user experiences inconvenience because the user has to browse pages to find the text items (e.g., nipple, axillary, lymph node, nodule) related to the breast examinations.

However, referring to an execution screen 320 illustrated in FIG. 3, when the ultrasound apparatus 100 receives an input of requesting an annotation setup interface from the user, the ultrasound apparatus 100 may identify a diagnostic field. Because the diagnostic field is the breast examination field 301, the ultrasound apparatus 100 may determine words to be text items related to the breast examination field 301, and may provide an annotation setup interface 300 including the determined text items that are most likely to be used for annotating ultrasound images during a breast examination. For example, the ultrasound apparatus 100 may provide the annotation setup interface 300 including Rt, Lt, Upper, Lower, Medial, Lateral, Nipple, Axillary, Lymph Node, Nodule, words (12h, 1h, 2h, . . . 11h, 1 cm, 2 cm, 3 cm, . . . 11 cm, +) which indicate measurement positions, or the like.

According to the present embodiment, the ultrasound apparatus 100 may group the text items according to semantic correlations. Then, the ultrasound apparatus 100 may arrange the grouped text items on respective rows.

For example, the ultrasound apparatus 100 may determine Rt (right) and Lt (left), which indicate respective breasts, to be included in a first group 301, and may arrange them on a first row. The ultrasound apparatus 100 may determine Upper, Lower, Medial, and Lateral, which indicate sectional directions, to be included in a second group 302, and may arrange them on a second row. The ultrasound apparatus 100 may determine Nipple, Axillary, Lymph Node, and Nodule, which indicate organs, to be included in a third group 303, and may arrange them on a third row. The ultrasound apparatus 100 may determine 12h, 1h, 2h, . . . 11h, 1 cm, 2 cm, 3 cm, . . . 11 cm, and +, which indicate measurement positions, to be included in a fourth group 304, and may arrange them on a fourth row. In this case, the user may rapidly generate an annotation by selecting a text item from each of rows on the annotation setup interface 300 in a sequential and downward direction, according to the semantic correlations.

In certain embodiments, the foregoing interface facilitates annotation of an ultrasound image while holding the ultrasound probe over the patient in one hand. Since the user is holding the ultrasound probe with one hand, which is most likely to be their favored hand, in certain embodiments facilitates easier and faster annotation of the ultrasound image by the user using one hand, which is likely to be their unfavored hand.

FIG. 4 is a diagram for describing an annotation setup interface that is provided according to a diagnostic field, according to an embodiment.

When the diagnostic field is general diagnostics in which a large number of abdomen ultrasound image scans occur, the ultrasound apparatus 100 may provide an annotation setup interface 410 including text items related to an abdomen, based on the diagnostic field (i.e., the general diagnostic center). For example, the annotation setup interface 410 that corresponds to the general diagnostic center may include the text items such as Rt, Lt, Transverse, Sagittal, Coronal, Proximal, Middle, Distal, Anterior, Posterior, Liver, Pancreas, Gallbladder, Spleen, IVC, Aorta, Kidney, Duodenum, or the like.

When the diagnostic field is orthopedic surgery in which a large number of musculoskeletal ultrasound image scans occur, the ultrasound apparatus 100 may provide an annotation setup interface 420 including text items related to a musculoskeletal system (MSK), based on the diagnostic field (i.e., the orthopedic surgery). For example, the annotation setup interface 420 that corresponds to the orthopedic surgery may include text items such as Rt, Lt, Middle, Distal, Posterior, Transverse, Sagittal, Coronal, Notch, ACL, MCL, Tuberosity, Bursa, Cartilage, Meniscus, Biceps Tendon, or the like.

When the diagnostic field is an endocrinology department in which a large number of thyroid ultrasound image scans occur, the ultrasound apparatus 100 may provide an annotation setup interface 430 including text items related to thyroid, based on the diagnostic department (i.e., the endocrinology department). For example, the annotation setup interface 430 that corresponds to the endocrinology department may include text items such as Rt, Lt, Upper, Lower, Medial, Lateral, Lobe, Isthmus, Lymph Node, CCA, IJV, Nodule, or the like.

When the diagnostic field is a cardiovascular department in which a large number of blood-vessel ultrasound image scans occur, the ultrasound apparatus 100 may provide an annotation setup interface 440 including text items related to a blood vessel, based on the diagnostic field (i.e., the cardiovascular department). For example, the annotation setup interface 440 that corresponds to the cardiovascular department may include text items such as Rt, Lt, Prox, Mid, Dist, CCA, ICA, Bulb, ECA, VA, SCA, IJV, Stenosis, Aneurysm, Graft, Anastomosis, or the like.

According to the present embodiment, the ultrasound apparatus 100 may adaptively change text items included in an annotation setup interface, depending on the diagnostic department, thereby allowing the user to conveniently set an annotation. Hereinafter, an operation of changing a layout of the annotation setup interface, the operation being performed by the ultrasound apparatus 100 to allow the user to efficiently set an annotation, will be described in detail.

FIG. 5 is a diagram for describing a method of determining a layout of an annotation setup interface, according to an embodiment, based on the tendencies of the user to make incorrect inputs.

In operation S510, the ultrasound apparatus 100 may obtain at least one of text item selecting pattern information and incorrect-input pattern information.

For example, the ultrasound apparatus 100 may analyze a pattern or a frequency with which text items are selected on a provided annotation setup interface. In this case, the ultrasound apparatus 100 may identify, based on a result of the analysis, that a first pattern in which an A-1 text item is selected from a first row, a B-1 text item is selected from a second row, and a C-1 text item is selected from a third row occurs the most, and a second pattern in which the A-1 text item is selected from the first row, a B-2 text item is selected from the second row, and the C-1 text item is selected from the third row occurs the second most.

The ultrasound apparatus 100 may obtain information about patterns of selecting text items according to respective users, patterns of selecting text items according to respective examinees, patterns of selecting text items according to respective probes, and patterns of selecting text items according to respective diagnostic departments.

According to the present embodiment, the ultrasound apparatus 100 may analyze the incorrect-input pattern information. For example, in a case where a pattern in which an user selects an A-1 text item from a first row, selects a B-1 text item from a second row, selects a C-1 text item from a third row, and then changes the B-1 text item selected from the second row to a B-2 text item occurs the most, the ultrasound apparatus 100 may determine that a probability in which the B-1 text item is incorrectly selected instead of the B-2 text item is high.

The ultrasound apparatus 100 may obtain information about patterns of incorrectly selecting text items according to respective users, patterns of incorrectly selecting text items according to respective examinees, patterns of incorrectly selecting text items according to respective probes, and patterns of incorrectly selecting text items according to respective diagnostic departments.

In operation S520, the ultrasound apparatus 100 may determine a layout of an annotation setup interface by using at least one of the text item selecting pattern information and the incorrect-input pattern information.

According to the present embodiment, the ultrasound apparatus 100 may emphatically display frequently-selected text items by using colors so as to emphasize the text items that are frequently selected on the annotation setup interface. For example, in a case where the A-1 text item, the B-1 text item, and a C-2 text item are frequently selected, the ultrasound apparatus 100 may determine the A-1 text item, the B-1 text item, and the C-2 text item to have a color different from that of other text items.

According to the present embodiment, the ultrasound apparatus 100 may determine a size of the frequently-selected text items to be relatively greater than that of other text items. The ultrasound apparatus 100 may determine a size of texts included the frequently-selected text items to be relatively larger than that of texts included in other text items.

According to the present embodiment, the ultrasound apparatus 100 may change an arrangement order or a size of text items, based on the incorrect-input pattern information. For example, in a case where the pattern in which the user selects the A-1 text item from the first row, selects the B-1 text item from the second row, selects the C-1 text item from the third row, and then changes the B-1 text item selected from the second row to the B-2 text item frequently occurs, the ultrasound apparatus 100 may change positions of the B-1 text item and the B-2 text item to each other, or may change a size of the B-2 text item to be greater than a size of the B-1 text item. In addition, the ultrasound apparatus 100 may determine a color of the B-1 text item to be similar to a background color.

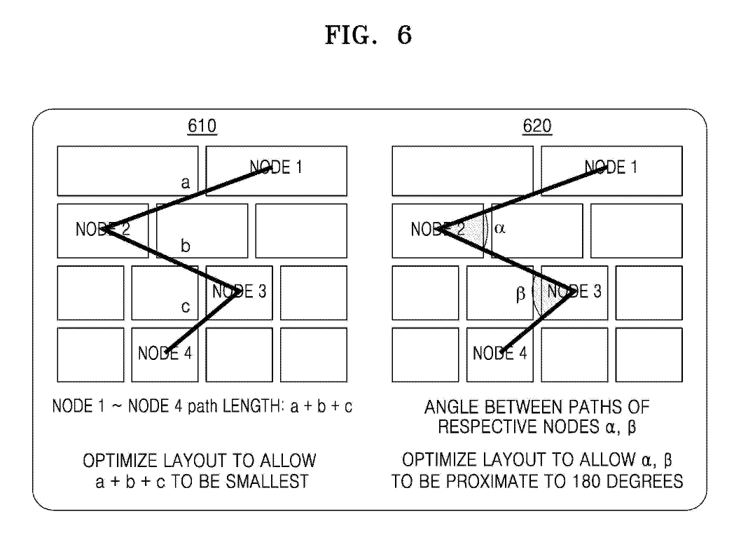

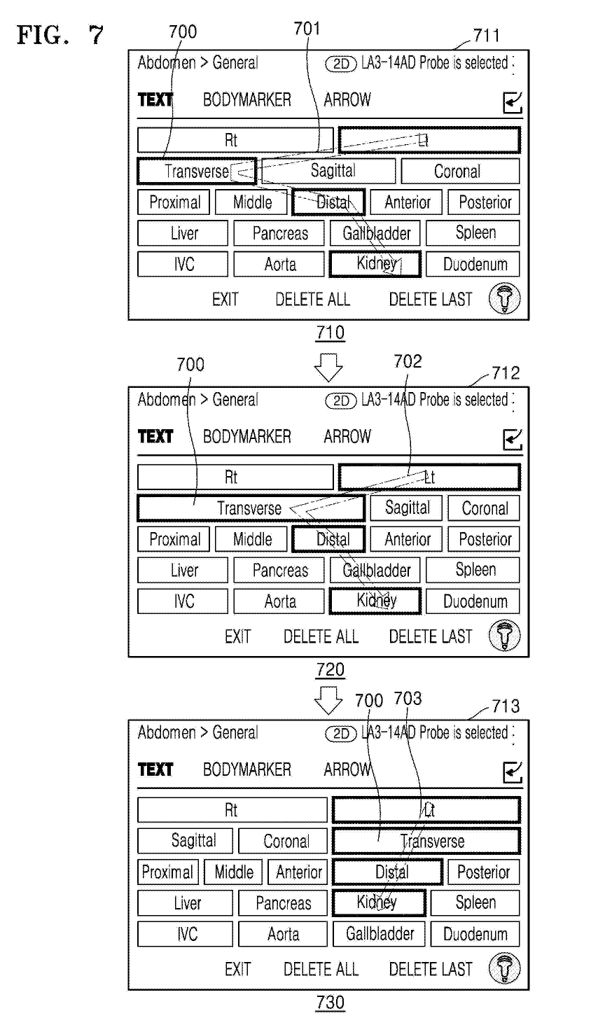

In operation S530, the ultrasound apparatus 100 may provide the annotation setup interface to the touchscreen 172, according to the determined layout. An operation, performed by the ultrasound apparatus 100, of optimizing a layout of the annotation setup interface will be described in detail with reference to FIGS. 6 and 7.

FIG. 6 is a diagram for describing an operation, performed by the ultrasound apparatus 100, of optimizing a layout of an annotation setup interface, according to an embodiment.

Referring to reference numeral 610 of FIG. 6, the ultrasound apparatus 100 may determine an arrangement of text items so as to allow a length of a line to be smallest, the line connecting text items of a pattern which are frequently selected by an user. For example, in a case where the text items which are frequently selected by the user are Node 1, Node 2, Node 3, and Node 4, the ultrasound apparatus 100 may determine a layout in which a position of Node 2 is moved to the right so that a length of a line (a+b+c) connecting Node 1 to Node 4 is decreased.

Referring to reference numeral 620 of FIG. 6, the ultrasound apparatus 100 may determine the arrangement of the text items so as to allow a total sum of angles to be close to 180 degrees, the angles being formed by the text items of the pattern which are frequently selected by the user. For example, in a case where the text items which are frequently selected by the user are Node 1, Node 2, Node 3, and Node 4, the ultrasound apparatus 100 may change the position of Node 2 to the right so as to allow a total sum of angles (α+β) to be proximate to 180 degrees, the angle α being formed by Node 1, Node 2, and Node 3, and the angle β being formed by Node 2, Node 3, and Node 4. An operation, performed by the ultrasound apparatus 100, of optimizing a layout of the annotation setup interface will be described in detail with reference to FIG. 7.

FIG. 7 is a diagram for describing an operation, performed by the ultrasound apparatus 100, of optimizing a layout of an annotation setup interface by using at least one of text item selecting pattern information and incorrect-input pattern information, according to an embodiment.

Referring to an execution screen 710 illustrated in FIG. 7, the ultrasound apparatus 100 may receive, from an user, a largest number of inputs with respect to a first pattern of selecting a Lt item, a Transverse item 700, a Distal item, and a Kidney item from a first annotation setup interface 711. Also, the ultrasound apparatus 100 may frequently receive an input with respect to a pattern in which a Sigittal item is incorrectly selected instead of the Transverse item 700 and then the Transverse item 700 is selected.

Referring to an execution screen 720 illustrated in FIG. 7, the ultrasound apparatus 100 may provide a second annotation setup interface 712 in which a size of the Transverse item 700 is increased to allow the user to further easily input the first pattern. In this case, a length of a line connecting the Lt item, the Transverse item 700, the Distal item, and the Kidney item is decreased from a first length 701 to a second length 702, thus, the user may easily select the first pattern on the second annotation setup interface 712, compared to the first annotation setup interface 711.

Referring to an execution screen 730 illustrated in FIG. 7, the ultrasound apparatus 100 may provide a third annotation setup interface 713 in which a length of the Transverse item 700 is adjusted to be increased to allow the user to further easily input the first pattern, and the Transverse item 700 is arranged at a right side. In this regard, the third annotation setup interface 713 may change a position of a line including the Kidney item and a position of a line including a Gallbladder item to each other. In this case, the length of the line connecting the Lt item, the Transverse item 700, the Distal item, and the Kidney item is decreased from the first length 701 to a third length 703, thus, an angle being formed by the Lt item, the Transverse item 700, the Distal item, and the Kidney item may become proximate to 180 degrees. Thus, the user may easily select the first pattern on the third annotation setup interface 713, compared to the first annotation setup interface 711, such that an incorrect-input rate with respect to annotations may be decreased.

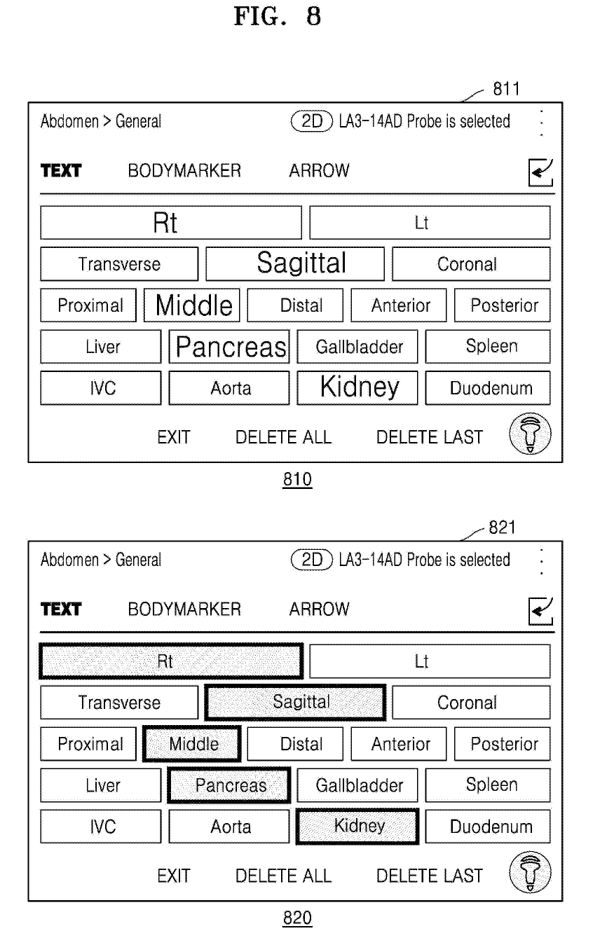

FIG. 8 is a diagram for describing an operation, performed by the ultrasound apparatus 100, of adjusting a size of a text included in a text item or adjusting a color of the text item, according to an embodiment.

With reference to FIG. 8, an example in which the ultrasound apparatus 100 receives a large number of inputs with respect to selecting an Rt item, a Sagittal, a Middle item, a Pancreas item, and a Kidney item from an annotation setup interface will now be described.

Referring to an execution screen 810 illustrated in FIG. 8, the ultrasound apparatus 100 may provide a first annotation setup interface 811 in which sizes of texts respectively included in the Rt item, the Sagittal, the Middle item, the Pancreas item, and the Kidney item, which are frequently selected, are relatively large. In this case, an user may further easily recognize the Sagittal, the Middle item, the Pancreas item, and the Kidney item on the first annotation setup interface 811.

Referring to an execution screen 820 illustrated in FIG. 8, the ultrasound apparatus 100 may provide a second annotation setup interface 821 in which a color of the Rt item, the Sagittal, the Middle item, the Pancreas item, and the Kidney item, which are frequently selected, is displayed differently from that of other items. For example, in the second annotation setup interface 821, the color of the Rt item, the Sagittal, the Middle item, the Pancreas item, and the Kidney item may be blue, and a color of other items may be gray.

According to another embodiment, the ultrasound apparatus 100 may determine a color (e.g., a blue color) of texts respectively included in the Rt item, the Sagittal, the Middle item, the Pancreas item, and the Kidney item to be different from a color (e.g., a black color) included in other items. Alternatively, the ultrasound apparatus 100 may determine a color (e.g., a blue color) of respective frames of the Rt item, the Sagittal, the Middle item, the Pancreas item, and the Kidney item to be different from a color (e.g., a black color) of frames of other items.

In this case, the user may further easily recognize the Rt item, the Sagittal, the Middle item, the Pancreas item, and the Kidney item on the second annotation setup interface 821.

FIG. 9 is a flowchart for describing a method, performed by the ultrasound apparatus 100, of adjusting a layout of an annotation setup interface by using examinee-related information, according to an embodiment.

In operation S910, the ultrasound apparatus 100 may receive an input of requesting an annotation setup interface. Operation S910 corresponds to operation S220 described with reference to FIG. 2, thus, detailed descriptions thereof are omitted here.

In operation S920, the ultrasound apparatus 100 may check examinee-related information about an examinee.

According to the present embodiment, the examinee-related information may be, but is not limited to, at least one of examinee's examination history information, examinee's examination posture information (the examinee lies supine or on his/her stomach, the examinee lies supine with his/her head turned to the right or left, the examinee lies with his/her head back, the examinee is sitting up, or the like, and examinee's physiological characteristic information (whether the examinee has kidneys, an age of the examinee, a gender of the examinee, or the like).

According to the present embodiment, an examination posture of the examinee may become different according to parts to be diagnosed. Thus, the ultrasound apparatus 100 may determine a detailed diagnostic target part, according to an examination posture of the examinee.

The ultrasound apparatus 100 may read the examinee-related information stored in a storage. Alternatively, the ultrasound apparatus 100 may receive the examinee-related information from an external server (e.g., a hospital server, or the like). Alternatively, the ultrasound apparatus 100 may receive an input of the examinee-related information from an user.

In operation S930, the ultrasound apparatus 100 may determine a layout of an annotation setup interface by using the examinee-related information.

According to the present embodiment, the ultrasound apparatus 100 may determine the layout of the annotation setup interface by using at least one of the examinee's examination history information, the examinee's examination posture information, and the examinee's physiological characteristic information. In certain embodiments, the layout can be determined to facilitate annotation of ultrasound images using only one hand, such as, while holding the ultrasound probe.

For example, the ultrasound apparatus 100 may determine the layout in which text items which are included in an annotation used in a previous examination are arranged with a priority or emphasized. Also, in a case where an examination posture of the examinee corresponds to a case where the examinee lies on his/her side and lifts up one leg, the ultrasound apparatus 100 may determine that a diagnosis target part is a prostate, and may determine a layout in which text items related to a prostate are arranged with a priority or emphasized.

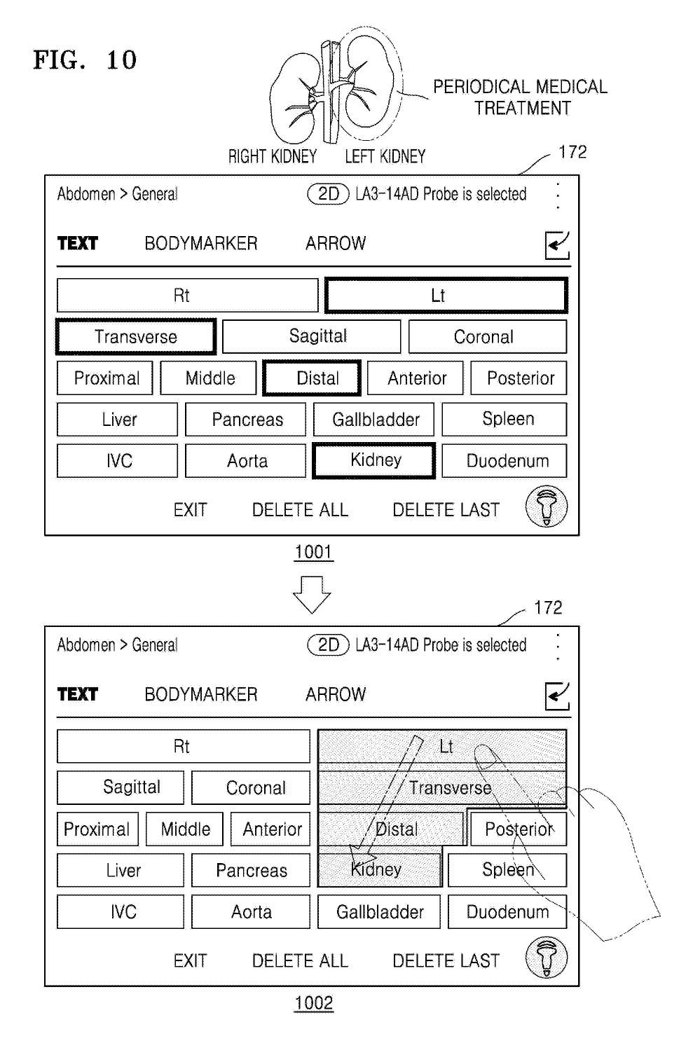

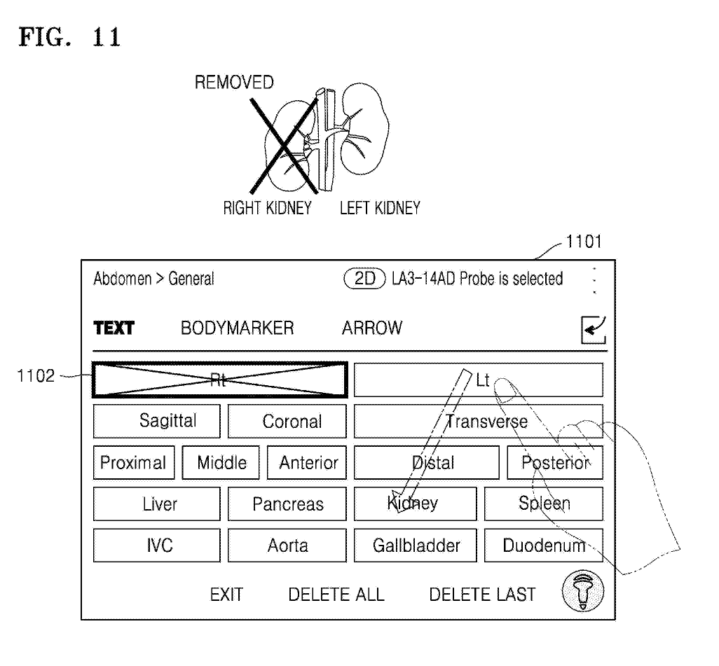

In operation S940, the ultrasound apparatus 100 may provide the annotation setup interface based on the determined layout. An operation, performed by the ultrasound apparatus 100, of providing an annotation setup interface according to examinee-related information will now be described in detail with reference to FIGS. 10 and 11.

FIG. 10 is a diagram for describing an operation, performed by the ultrasound apparatus 100, of providing an annotation setup interface by using an examination history of an examinee, according to an embodiment.

Referring to an execution screen 1001 illustrated in FIG. 10, the ultrasound apparatus 100 may check examinee-related information and thus may recognize that the examinee periodically receives a medical treatment for a left kidney, and in a previous examination, an Lt item, a Transverse item, a Distal item, and a Kidney item were selected from an annotation setup interface such that an annotation was stored as ‘LT Transverse Distal Kidney’. Accordingly, the execution screen may present an interface for annotating ultrasound images of the kidney, including text items that are commonly used to describe ultrasound images of the kidney. Moreover, the execution screen may also be arranged such that commonly “LT” (Left), “Transverse”, “Distal”, and “Kidney” are arranged in manner that are easier to select, such as being proximate to each other.

Referring to an execution screen 1002 illustrated in FIG. 10, in order to allow a user to easily select the Lt item, the Transverse item, the Distal item, and the Kidney item, based on the examination history of the examinee, the ultrasound apparatus 100 may provide an annotation setup interface in which the Lt item, the Transverse item, the Distal item, and the Kidney item are arranged at a right side. According to the present embodiment, the ultrasound apparatus 100 may emphasize the Lt item, the Transverse item, the Distal item, and the Kidney item on the annotation setup interface.

FIG. 11 is a diagram for describing an operation, performed by the ultrasound apparatus 100, of providing an annotation setup interface by using physiological characteristic information of an examinee, according to an embodiment.

Referring to FIG. 11, when the ultrasound apparatus 100 receives an input of requesting the annotation setup interface, the ultrasound apparatus 100 may check the physiological characteristic information of the examinee. Based on the physiological characteristic information of the examinee, the ultrasound apparatus 100 may recognize that a right kidney of the examinee has been removed and the examinee periodically receives a medical treatment for a left kidney. In this case, the ultrasound apparatus 100 may perform a disable display 1102 on an Rt item so as to prevent an user from incorrectly selecting the Rt item on an annotation setup interface 1101.

FIG. 12 is a flowchart for describing a method, performed by the ultrasound apparatus 100, of adjusting a layout of an annotation setup interface by using user-related information, according to an embodiment.

In operation S1210, the ultrasound apparatus 100 may receive an input of requesting an annotation setup interface. Operation S1210 corresponds to operation S220 described with reference to FIG. 2, thus, detailed descriptions thereof are omitted here.

In operation S1220, the ultrasound apparatus 100 may check user-related information.

According to the present embodiment, the user-related information may include, but is not limited to, information about with which hand the user uses the probe 20 (e.g., left handed, or right handed), information about a pattern by which the user selects a text item (e.g., information about an user-preferred selection pattern), information about a posture of the user (e.g., information about the user who is sitting at the right side behind the ultrasound apparatus 100), or the like.

The ultrasound apparatus 100 may read the user-related information stored in a storage. Alternatively, the ultrasound apparatus 100 may receive the user-related information from an external server (e.g., a hospital server, or the like). Alternatively, the ultrasound apparatus 100 may receive an input of the user-related information from the user.

In operation S1230, the ultrasound apparatus 100 may determine a layout of the annotation setup interface by using the user-related information.

According to the present embodiment, the ultrasound apparatus 100 may determine the layout of the annotation setup interface by using at least one of the information about with which hand the user uses the probe 20, the information about the pattern by which the user selects a text item, and the information about the posture of the user. For example, in a case where the user is right-handed, there is a high probability that the user usually holds the probe 20 in a right hand and manipulates the touchscreen 172 with a left hand. Thus, as a result of checking the user-related information, when the user is right-handed, the ultrasound apparatus 100 may determine an arrangement of text items so as to allow the user to conveniently select text items by using the left hand.

Also, as the result of checking the user-related information, when the user prefers a pattern in which the user selects an A-1 item, selects a B-2 item, and then selects a C-2 item, the ultrasound apparatus 100 may determine the layout of the annotation setup interface so as to allow the user to conveniently select the A-1 item, the B-2 item, and the C-2 item sequentially with the left hand.

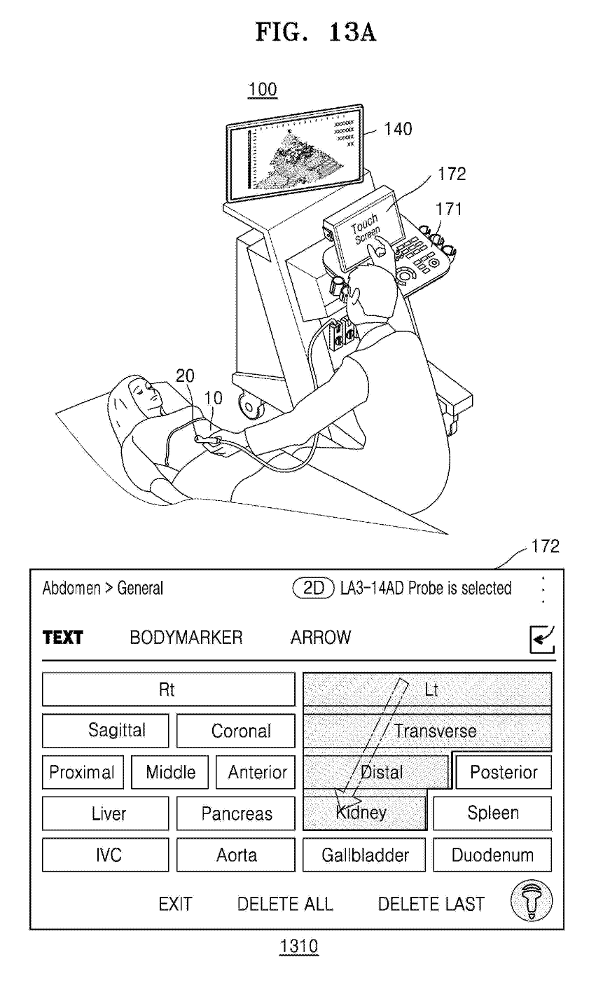

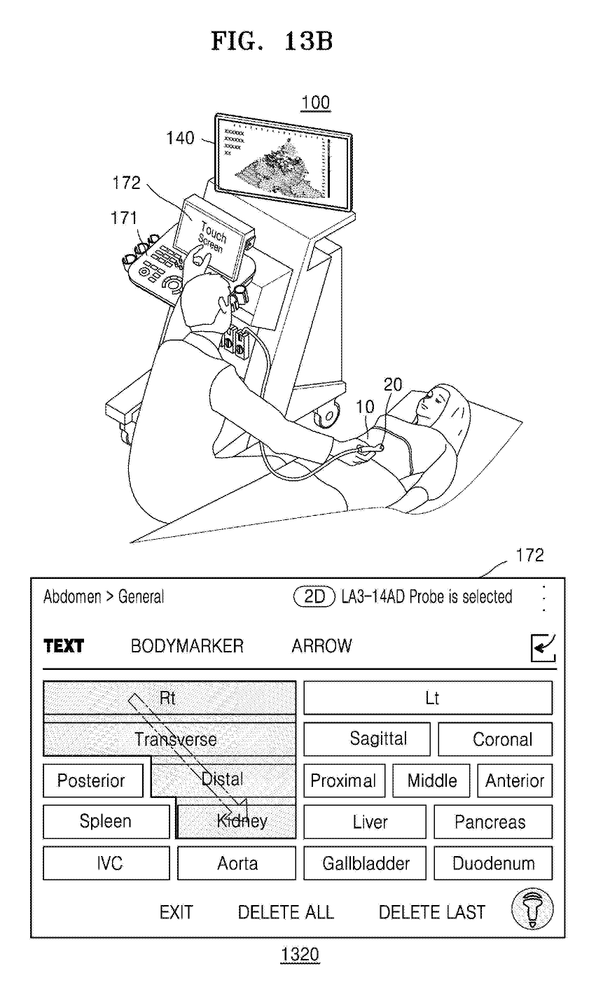

In operation S1240, the ultrasound apparatus 100 may provide the annotation setup interface based on the determined layout to the touchscreen 172. An operation, performed by the ultrasound apparatus 100, of providing an annotation setup interface according to the user-related information will be further described with reference to FIGS. 13A and 13B.

FIGS. 13A and 13B are diagrams for describing an operation, performed by the ultrasound apparatus 100, of determining a layout of an annotation setup interface according to by which hand an user manipulates a control panel, according to an embodiment.

With reference to FIGS. 13A and 13B, it is assumed that the user frequently selects a Lt item, a Transverse item, a Distal item, and a Kidney item with respect to an ultrasound image of the examinee 10.

Referring to FIG. 13A, in a case where an input of requesting an annotation setup interface is received from the user, the ultrasound apparatus 100 may check user-related information. As a result of checking the user-related information, when it is determined that the user is left-handed, the ultrasound apparatus 100 may determine a layout of an annotation setup interface 1310 so as to allow the user to easily select text items with a right hand. For example, the Lt item, the Transverse item, the Distal item, and the Kidney item may be arranged on the annotation setup interface 1310 such that the Lt item, the Transverse item, the Distal item, and the Kidney item may be selected at one time in response to a drag-down input in a direction from an upper right side to a lower left side.

Referring to FIG. 13B, as a result of checking the user-related information, when it is determined that the user is right-handed, the ultrasound apparatus 100 may determine a layout of an annotation setup interface 1320 so as to allow the user to easily select text items with a left hand. For example, the Lt item, the Transverse item, the Distal item, and the Kidney item may be arranged on the annotation setup interface 1320 such that the Lt item, the Transverse item, the Distal item, and the Kidney item may be selected at one time in response to a drag-down input in a direction from an upper left side to a lower right side.

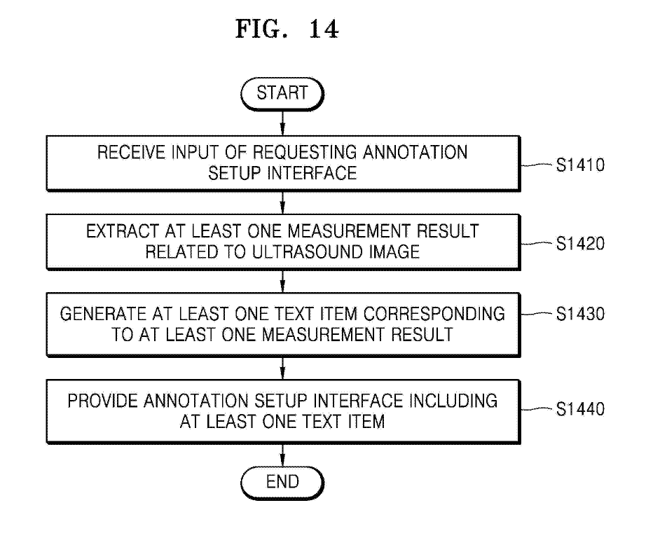

FIG. 14 is a flowchart for describing a method, performed by the ultrasound apparatus 100, of providing an annotation setup interface by using measurement result information, according to an embodiment.

In operation S1410, the ultrasound apparatus 100 may receive an input of requesting an annotation setup interface. Operation S1410 corresponds to operation S220 described with reference to FIG. 2, thus, detailed descriptions thereof are omitted here.

In operation S1420, the ultrasound apparatus 100 may extract at least one measurement result related to an ultrasound image.

According to the present embodiment, the ultrasound apparatus 100 may read, from a storage, measurement values obtained in an ultrasound diagnosis process with respect to an examinee. Alternatively, the ultrasound apparatus 100 may receive, from a server (e.g., a hospital server, a cloud server, or the like), the measurement values obtained in the ultrasound diagnosis process with respect to the examinee.

In operation S1430, the ultrasound apparatus 100 may generate at least one text item corresponding to the at least one measurement result.

For example, in a case where a size of a lesion measured in the ultrasound diagnosis process with respect to the examinee is 3 cm, 4.5 cm, and 3.8 cm, respectively, the ultrasound apparatus 100 may generate text items that respectively correspond to 3 cm, 4.5 cm, and 3.8 cm.

According to the present embodiment, in a case where the number of the measurement values is equal to or greater than a threshold value (e.g. 5), the ultrasound apparatus 100 may generate a predetermined number of measurement values as text items, based on measurement time information. For example, in a case where the measurement values are obtained in order of 2.9 cm, 3 cm, 3.5 cm, 3.8 cm, 3.2 cm, 3.1 cm, and 3.3 cm, the ultrasound apparatus 100 may generate only 3.3 cm, 3.1 cm, and 3.2 cm which are the latest values, as the text items.

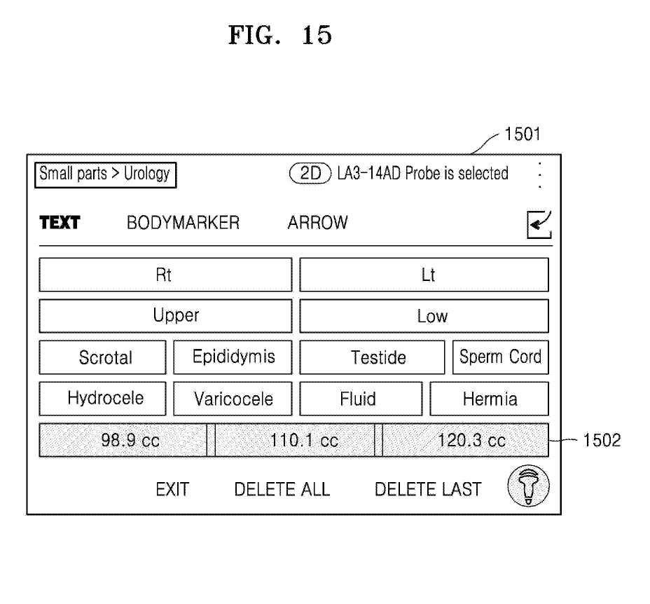

In operation S1440, the ultrasound apparatus 100 may provide an annotation setup interface including the at least one text item corresponding to the at least one measurement result. In this case, an user may conveniently generate the measurement result as an annotation. An operation, performed by the ultrasound apparatus 100, of providing the annotation setup interface by using the measurement result, will be further described with reference to FIG. 15.

FIG. 15 is a diagram for describing an operation, performed by the ultrasound apparatus 100, of determining text items by using measurement result information, according to an embodiment. With reference to FIG. 15, it is assumed that an examinee is a male patient in his fifties for whom a volume of a prostate has been measured by using a probe for an inner examination.

Referring to FIG. 15, the ultrasound apparatus 100 may receive, from an user, an input of requesting an annotation setup interface 1501. In this case, the ultrasound apparatus 100 may check whether measurement values obtained with respect to an ultrasound image exist. As a result of the check, the ultrasound apparatus 100 may recognize that previous measurement values of 98.9 cc, 110.1 cc, and 120.3 cc which were obtained by measuring the volume of the prostate exist.

The ultrasound apparatus 100 may generate 98.9 cc, 110.1 cc, and 120.3 cc items 1502 by using the measurement values, and may arrange the 98.9 cc, 110.1 cc, and 120.3 cc items 1502 on a fifth row of the annotation setup interface 1501. In this case, the user may select an appropriate measurement value from among the 98.9 cc, 110.1 cc, and 120.3 cc items 1502 and may generate the selected measurement value as an annotation.

The ultrasound apparatus 100 may determine text items according to information about a hospital in which the ultrasound apparatus 100 is installed. An operation, performed by the ultrasound apparatus 100, of providing an annotation setup interface according to the information about a hospital in which the ultrasound apparatus 100 is installed will now be described in detail with reference to FIG. 16.

FIG. 16 is a diagram for describing an operation, performed by the ultrasound apparatus 100, of providing an annotation setup interface by using information about the known specialty of the hospital in which the ultrasound apparatus 100 is installed, according to an embodiment.

Referring to a first annotation setup interface 1610 illustrated in FIG. 16, the hospital in which the ultrasound apparatus 100 is installed may be a senior-dedicated hospital 1601. In this case, the ultrasound apparatus 100 may provide the first annotation setup interface 1610 including text items related to degenerative tissues which mostly occur to old people. For example, rheumarthritis mostly occurs old people, thus, text items related to an infection and a bloodstream may be arranged in the first annotation setup interface 1610. Alternatively, the ultrasound apparatus 100 may determine the first annotation setup interface 1610 to include text items related to an in-depth joint ultrasound examination.

Referring to a second annotation setup interface 1620 illustrated in FIG. 16, the hospital in which the ultrasound apparatus 100 is installed may be an orthopedic hospital 1602. In a case where the in-depth joint ultrasound examination is mainly performed in the orthopedic hospital 1602, the ultrasound apparatus 100 may provide the second annotation setup interface 1620 including text items related to large and small ligaments and/or joints of shoulders, elbows, wrists, hands, hip joints, knees, ankles, feet, or the like. Also, in a case where a soft-tissue ultrasound examination is mainly performed in the orthopedic hospital 1602, the ultrasound apparatus 100 may provide the second annotation setup interface 1620 including text items related to a method of examining muscles and skins of arms, legs, and torsos.

Referring to a third annotation setup interface 1630 illustrated in FIG. 16, the hospital in which the ultrasound apparatus 100 is installed may be a kidney-dedicated hospital 1603. Because there are many gout cases due to an uric acid with respect to patients having a high liver somatic index, when an input of requesting an annotation setup interface is input, the ultrasound apparatus 100 may provide the third annotation setup interface 1630 in which text items related to an uric acid or a gout are arranged on an entire page.

According to the present embodiment, an annotation setup interface may be adaptively provided depending on types of hospital in which the ultrasound apparatus 100 is installed, thus, an user may efficiently set an annotation by using the annotation setup interface. Hereinafter, a method of inputting, by an user, an annotation by using an annotation setup interface will be described in detail.

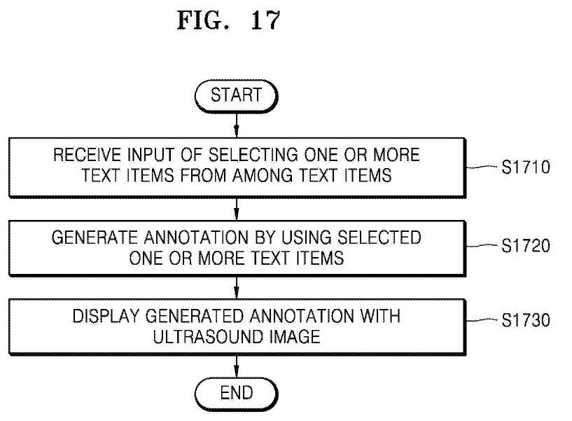

FIG. 17 is a flowchart for describing a method, performed by the ultrasound apparatus 100, of generating an annotation, based on an input, according to an embodiment.

In operation S1710, the ultrasound apparatus 100 may receive an input of selecting one or more text items from among text items included in an annotation setup interface.

According to the present embodiment, the input of selecting the one or more text items may include, but are not limited to, an input (e.g., a tap input, a double-tap input, or the like) of touching the one or more text items, or a drag input for connecting the one or more text items. The drag input may be referred to as a swipe input.

For example, an user may select one or more text items from among a plurality of text items displayed on an annotation setup interface so as to generate the one or more text items as an annotation.

In operation S1720, the ultrasound apparatus 100 may generate the annotation by using the selected one or more text items.

According to the present embodiment, the ultrasound apparatus 100 may generate the annotation by connecting texts included in the selected one or more text items.

In operation S1730, the ultrasound apparatus 100 may display both the generated annotation and an ultrasound image on the display 140.

According to the present embodiment, the ultrasound apparatus 100 may display the generated annotation on the ultrasound image. Alternatively, the ultrasound apparatus 100 may partially overlap the annotation with the ultrasound image, or may display the annotation on a portion of the annotation setup interface, wherein the portion does not overlap with the ultrasound image.

The ultrasound apparatus 100 may store the annotation mapped with the ultrasound image in a storage. Alternatively, the ultrasound apparatus 100 may transmit the annotation mapped with the ultrasound image to an external server (e.g., a hospital server or a personal server).

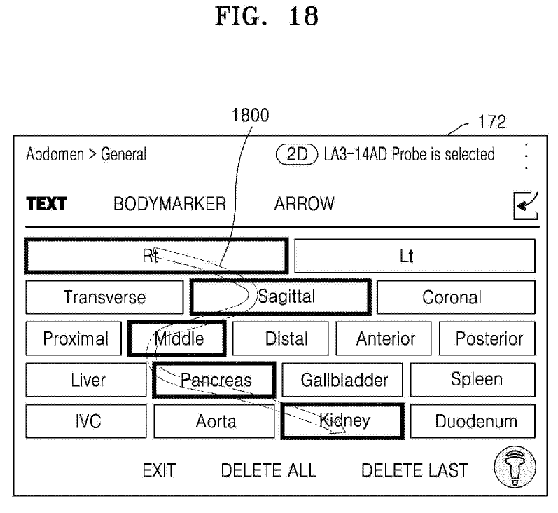

FIG. 18 is a diagram for describing an operation, performed by the ultrasound apparatus 100, of providing an image for guiding a method of setting an annotation, according to an embodiment.

According to the present embodiment, the ultrasound apparatus 100 may provide a guide image 1800 for guiding a method of setting an annotation on an annotation setup interface. For example, in a case where the annotation setup interface provides a function of setting an annotation, based on a drag input (e.g., one touch drag-dawn interaction), the ultrasound apparatus 100 may display the guide image 1800 for inducing a drag input to select a plurality of text items included in the annotation setup interface.

In this case, an user may conveniently select, by one drag input, an Rt item, a Sagittal item, a Middle item, a Pancreas item, and a Kidney item by referring to the guide image 1800.

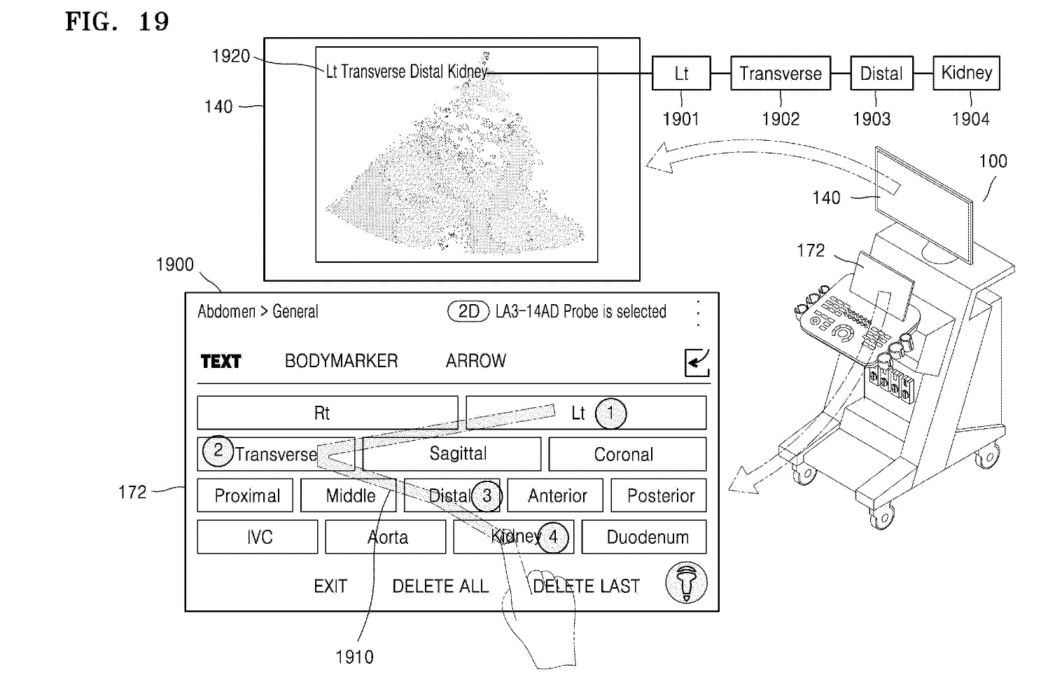

FIG. 19 is a diagram for describing an operation, performed by the ultrasound apparatus 100, of generating an annotation, based on an input received via an annotation setup interface 1900, according to an embodiment.

Referring to FIG. 19, the ultrasound apparatus 100 may provide the annotation setup interface 1900 on the touchscreen 172. For example, the ultrasound apparatus 100 may provide the annotation setup interface 1900 on which an Rt item and an Lt item are displayed on a first row, a Transverse item, a Sagittal item, and a Coronal item are displayed on a second row, a Proximal item, a Middle item, a Distal item, an Anterior item, and a Posterior item are displayed on a third row, and an IVC item, an Aorta item, a Kidney item, and a Duodenum item are displayed on a fourth row.

The ultrasound apparatus 100 may receive a drag input 1910 of finger-dragging on the annotation setup interface 1900. In this regard, the ultrasound apparatus 100 may select text items from respective rows by analyzing a path of the drag input 1910. For example, the ultrasound apparatus 100 may select, from the respective rows, the Lt item, the Transverse item, the Distal item, and the Kidney item which most overlap the path of the drag input 1910.

The ultrasound apparatus 100 may generate an annotation by connecting texts included in the text items selected from the respective rows. For example, the ultrasound apparatus 100 may generate an annotation ‘Lt Transverse Distal Kidney’ 1920 by connecting Lt 1901, Transverse 1902, Distal 1903, and Kidney 1904. In this regard, the generated annotation may be displayed with an ultrasound image on the display 140.

According to the present embodiment, an user may collectively select text items with an one-touch drag down input on the annotation setup interface 1900, e.g., items Lt 1901, Transverse 1902, Distal 1903 and Kidney 1904 appear vertically in line, such that the user may easily set an annotation with one hand while the user holds the probe 20 in the other hand.

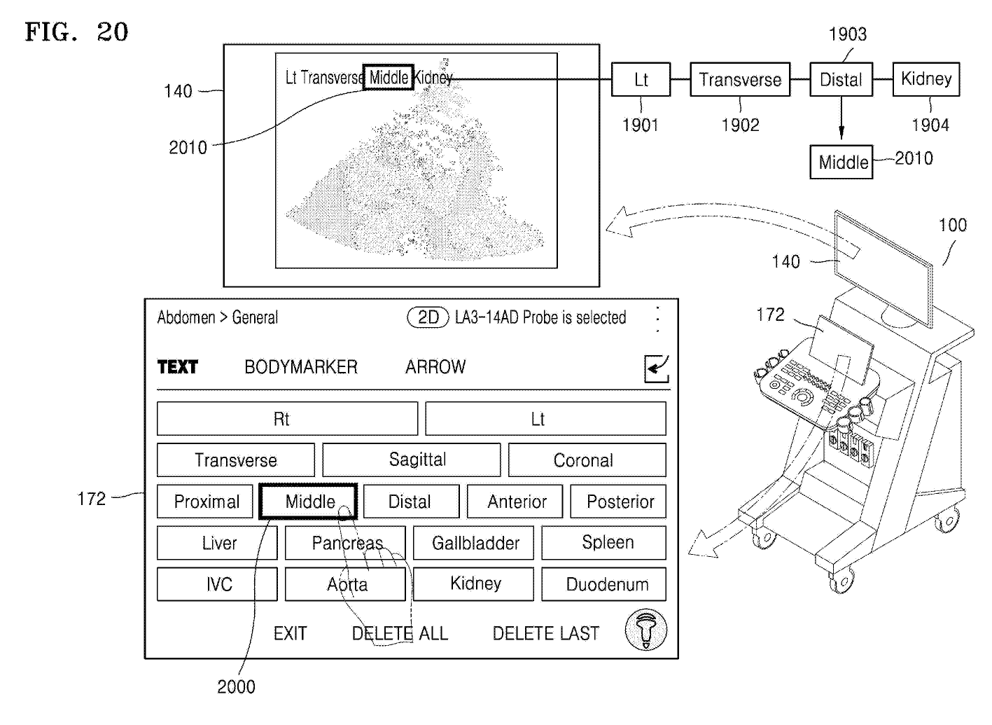

FIG. 20 is a diagram for describing an operation, performed by the ultrasound apparatus 100, of amending an annotation, based on an input received via an annotation setup interface, according to an embodiment. With reference to FIG. 20, it is assumed that an user attempts to amend Distal to Middle.

Referring to FIG. 20, the user may select a Lt item, a Transverse item, a Distal item, and a Kidney item with a one-touch drag down input, and may touch a Middle item 2000 on a third row. Because only one item can be selected from a row for an annotation the ultrasound apparatus 100 may determine that an input of changing selection of the Distal item to selection of the Middle item 2000 has been received. Thus, the ultrasound apparatus 100 may generate a new annotation ‘Lt Transverse Middle Kidney’ 2010 by changing Distal to Middle in the annotation ‘Lt Transverse Distal Kidney’ 1920, and may display the new annotation 2010 with the ultrasound image.

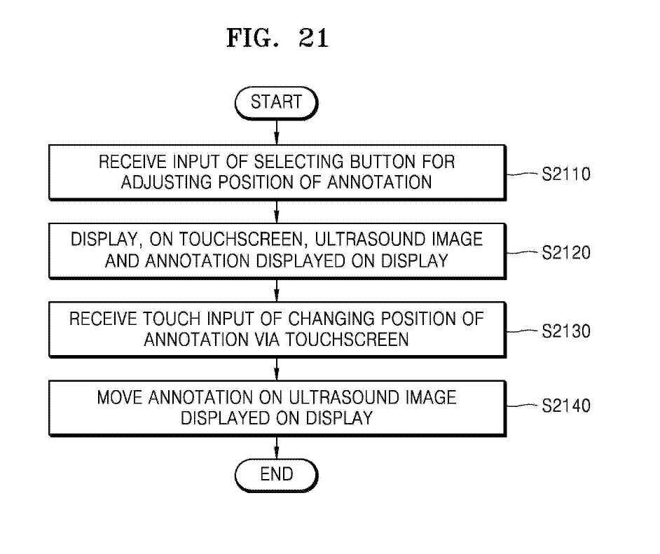

FIG. 21 is a flowchart for describing a method, performed by the ultrasound apparatus 100, of moving a position of an annotation, in response to a touch input, according to an embodiment.

In operation S2110, the ultrasound apparatus 100 may receive an input of selecting a button for adjusting a position of an annotation displayed on an ultrasound image. The button for adjusting a position of an annotation may be, but is not limited to, a hardware button included in the control panel 171 or a software button displayed on the touchscreen 172.

According to the present embodiment, in a case where an user selects the button for adjusting a position of an annotation, the ultrasound apparatus 100 may change an operation mode to an annotation position editing mode.

In operation S2120, the ultrasound apparatus 100 may display, on the touchscreen 172, an ultrasound image and an annotation displayed on the display 140.

According to the present embodiment, in response to the input of selecting the button for adjusting a position of an annotation, an execution screen of the touchscreen 172 may be synchronized with an execution screen of the display 140.

In operation S2130, the ultrasound apparatus 100 may receive a touch input of changing a position of an annotation with respect to the ultrasound image via the touchscreen 172. For example, the ultrasound apparatus 100 may receive a drag input of dragging the annotation from a first position to a second position.

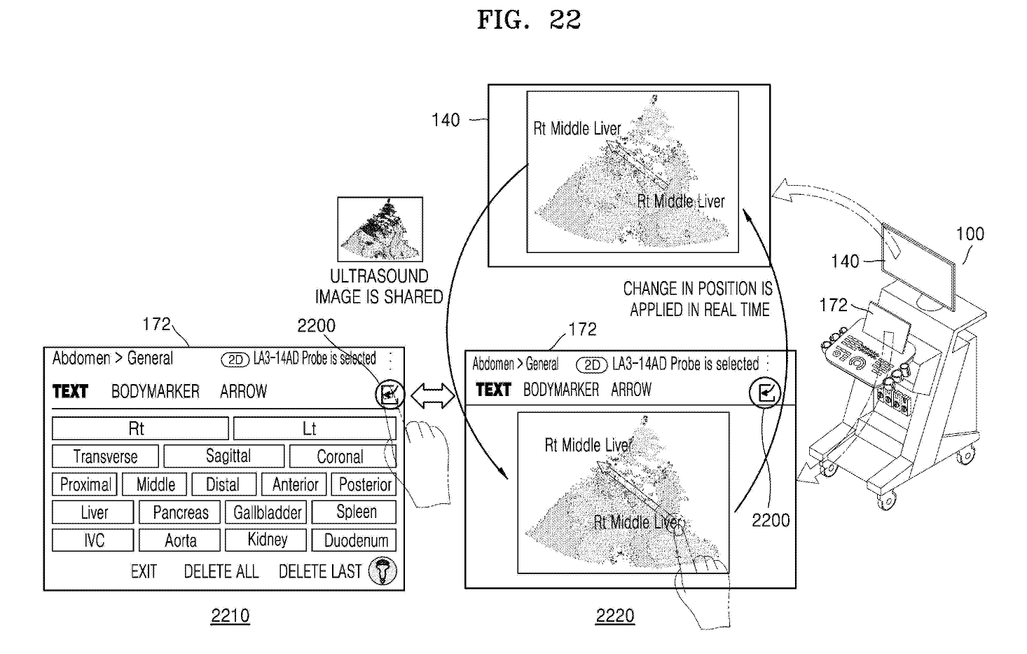

In operation S2140, the ultrasound apparatus 100 may move the annotation displayed on the display 140, in response to the touch input. An operation, performed by the ultrasound apparatus 100, of changing a position of an annotation, in response to an input from an user, will be described in detail with reference to FIG. 22.

FIG. 22 is a diagram for describing an operation, performed by the ultrasound apparatus 100, of receiving an input of moving a position of an annotation via the touchscreen 172, according to an embodiment.

Referring to FIG. 22, the ultrasound apparatus 100 may display both an annotation of Rt Middle Liver and an ultrasound image on the display 140. The annotation may be positioned at a lower right portion (a first position) of the ultrasound image.

Referring to an execution screen 2210 illustrated in FIG. 22, in order to adjust a position of the annotation, an user may touch a position editing button 2200 on the touchscreen 172 on which an annotation setup interface is displayed.

Referring to an execution screen 2220 illustrated in FIG. 22, when an input of touching the position editing button 2200 is received, the ultrasound apparatus 100 may enter a position editing mode, and may synchronize an execution screen of the touchscreen 172 with an execution screen of the display 140. For example, the ultrasound apparatus 100 may display, on the touchscreen 172, the ultrasound image including the annotation, instead of the annotation setup interface. The ultrasound apparatus 100 may receive, from the user via the touchscreen 172, an input of dragging the annotation from a lower right portion of the ultrasound image to an upper left portion of the ultrasound image.

When the drag input of dragging the annotation is received via the touchscreen 172, the ultrasound apparatus 100 may move a position of the annotation displayed on the display 140, in response to the drag input. For example, the ultrasound apparatus 100 may move the position of the annotation displayed on the display 140 from the lower right portion (the first position) of the ultrasound image to an upper left portion (a second position) of the ultrasound image.

According to the present embodiment, when the user moved the annotation to a desired position, the user may select again the position editing button 2200. In this regard, the ultrasound apparatus 100 may end the position editing mode, and may display again the annotation setup interface on the touchscreen 172 as illustrated in the execution screen 2210 illustrated in FIG. 22.

According to the present embodiment, the user may easily adjust a position of an annotation by using a drag input with one hand while the user holds the probe 20 in the other hand.

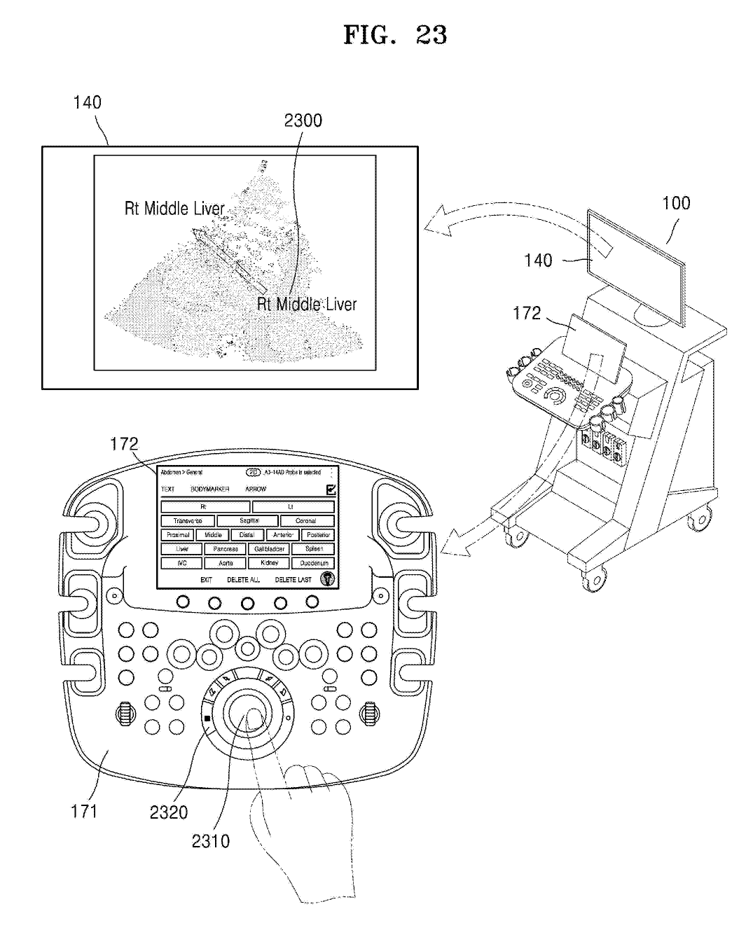

FIG. 23 is a diagram for describing an operation, performed by the ultrasound apparatus 100, of receiving an input of moving a position of an annotation 2300 by using a trackball 2310 in the control panel 171, according to an embodiment.

Referring to FIG. 23, the ultrasound apparatus 100 may receive an input of adjusting the position of the annotation 2300 by using the trackball 2310 included in the control panel 171. For example, an user may move, by using the trackball 2310, the position of the annotation 2300 displayed on the display 140 from a lower right portion (a first position) of an ultrasound image to an upper left portion (a second position) of the ultrasound image. When the annotation 2300 is moved to a desired position, the user may select a finish button (e.g., a set button) 2320.

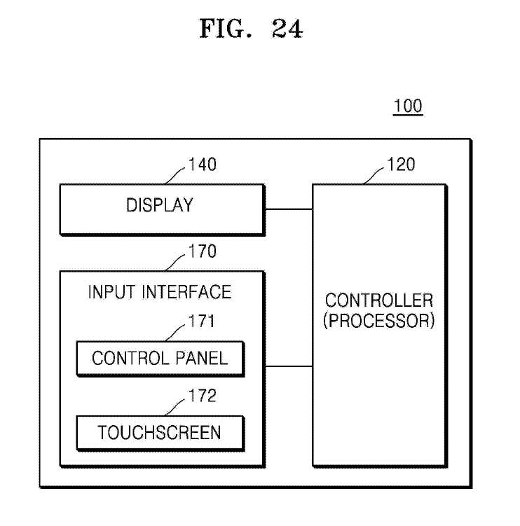

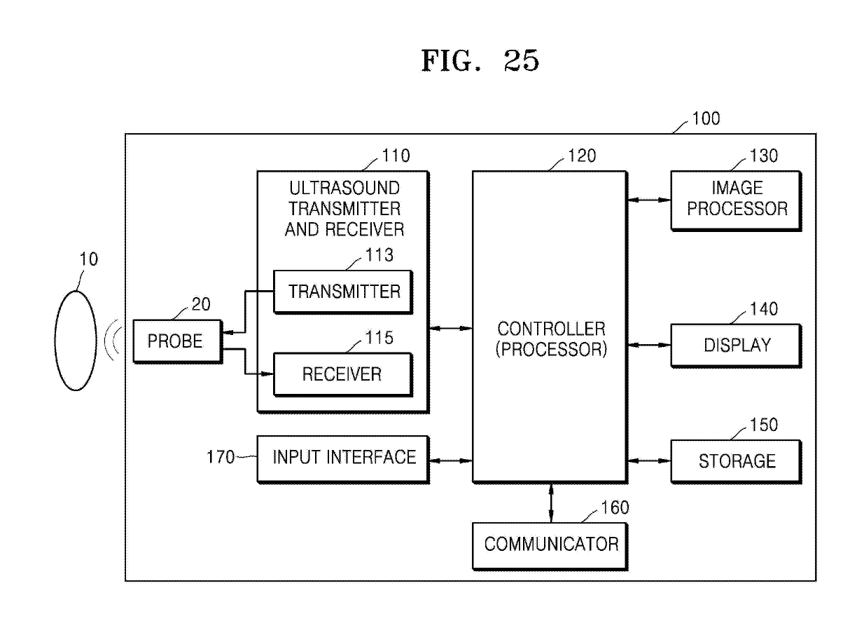

FIGS. 24 and 25 are block diagrams for describing configurations of the ultrasound apparatus 100, according to embodiments.

As illustrated in FIG. 24, the ultrasound apparatus 100 may include a controller (a processor) 120, the display 140, and the input interface 170 including the control panel 171 and the touchscreen 172. However, not all elements shown in FIG. 24 are necessary elements. The ultrasound apparatus 100 may be embodied with more elements than the shown elements or may be embodied with fewer elements than the shown elements.

The input interface 170 may be configured to facilitate annotation of ultrasound images by displaying useful text information with a single hand, while the other hand is holding the probe as described above.

For example, as illustrated in FIG. 25, the ultrasound apparatus 100 may further include the probe 20, an ultrasound transmitter and receiver 110, an image processor 130, a storage 150, and a communications interface 160, in addition to the controller 120, the display 140, and the input interface 170. The elements will now be sequentially described.

The ultrasound apparatus 100 may be a cart-type ultrasound apparatus or a portable-type ultrasound apparatus, which is portable, moveable, mobile, or hand-held. Examples of the portable-type ultrasound apparatus 100 may include, but is not limited to, a smart phone, a laptop computer, a personal digital assistant (PDA), and a tablet personal computer (PC), each of which may include the probe 20 and a software application.

The probe 20 may include a plurality of transducers. The plurality of transducers may transmit ultrasound signals to the examinee 10, in response to transmitting signals received by the probe 20 from a transmitter 113. The plurality of transducers may receive ultrasound signals reflected from the examinee 10 so as to generate reception signals. In addition, the probe 20 and the ultrasound apparatus 100 may be formed in one body (e.g., disposed in a single housing), or the probe 20 and the ultrasound apparatus 100 may be formed separately (e.g., disposed separately in separate housings) but linked to each other in a wired or wireless manner. In addition, the ultrasound apparatus 100 may include one or more probes 20 according to embodiments.

The controller 120 may control the transmitter 113 to generate transmitting signals to be applied to each of the plurality of transducers, based on positions and focal points of the plurality of transducers included in the probe 20.

The controller 120 may control a receiver 115 to generate ultrasound data by converting reception signals received from the probe 20 from analogue to digital signals and summing the reception signals converted into a digital form, based on positions and focal points of the plurality of transducers.

The image processor 130 may generate an ultrasound image by using the ultrasound data generated by the receiver 115.

The display 140 may display the generated ultrasound image and a plurality of pieces of information processed by the ultrasound apparatus 100. The ultrasound apparatus 100 may include one or more displays 140 according to embodiments. The display 140 may include a touchscreen in combination with a touch panel.

The controller 120 may control the operations of the ultrasound apparatus 100 and flow of signals between the internal elements of the ultrasound apparatus 100. The controller 120 may include a memory that stores a program or data to perform functions of the ultrasound apparatus 100, and a processor and/or a microprocessor (not shown) configured to process the program or data. For example, the controller 120 may control the operation of the ultrasound apparatus 100 by receiving a control signal from the input interface 170 or an external apparatus.

The controller 120 may determine text items, based on a diagnostic department that corresponds to an ultrasound image. In addition, the controller 120 may determine text items by using measurement result information related to the ultrasound image. The controller 120 may determine text items, based on at least one of a type of a probe, a function used in obtaining the ultrasound image, and information about a hospital in which the ultrasound apparatus 100 is installed.

The controller 120 may provide an annotation setup interface including the determined text items via the touchscreen 172. In this regard, the controller 120 may group the text items, based on semantic correlations, and may arrange the grouped text items on respective rows.

The controller may determine a layout of the annotation setup interface by using at least one of examinee-related information and user-related information. The controller 120 may determine, by using at least one of text item selecting pattern information and incorrect-input pattern information, at least one of positions of the text items included in the annotation setup interface, sizes of the text items, colors of the text items, fonts of texts included in the text items, sizes of the texts, and colors of the texts.

The controller 120 may receive, via the touchscreen 172, an input of selecting at least one text item from among the text items. The controller 120 may generate an annotation by using the selected at least one text item, and may control the display 140 to display the generated annotation on the ultrasound image.

In a case where an input of selecting a button for adjusting a position of the annotation displayed on the ultrasound image is received, the controller 120 may display, on the touchscreen 172, the ultrasound image and the annotation displayed on the display 140. The controller 120 may receive a touch input of changing the position of the annotation with respect to the ultrasound image via the touchscreen 172, and may move the annotation displayed on the display 140, in response to the touch input.

The ultrasound apparatus 100 may include the communicator 160, and may be connected to external apparatuses, for example, servers, medical apparatuses, and portable devices such as smart phones, tablet personal computers (PCs), wearable devices, etc., via the communicator 160.

The communications interface 160 may include at least one element capable of communicating with the external apparatuses. For example, the communications interface 160 may include at least one of a short-range communication module, a wired communication module, and a wireless communication module.

The communications interface 160 may receive a control signal or data from an external apparatus and may transmit a control signal or data to the controller 120 so that the controller 120 may control the ultrasound apparatus 100 in response to the received control signal.

The storage 150 may store various types of data or programs for driving and controlling the ultrasound apparatus 100, input and/or output ultrasound data, obtained ultrasound images, or the like. For example, the storage 150 may store information about a pattern through which an user selects text items, incorrect-input pattern information, annotation information, or the like.

The input interface 170 may receive a user input to control the ultrasound apparatus 100 and may include a keyboard, a button, a keypad, a mouse, a trackball, a jog switch, a knob, a touchpad, a touch screen, a microphone, a motion input means, a biometrics input means, or the like. For example, the user input may include, but is not limited to, inputs for manipulating buttons, keypads, mice, trackballs, jog switches, or knobs, inputs for touching a touchpad or a touch screen, a voice input, a motion input, and a bioinformation input, for example, iris recognition or fingerprint recognition.

The embodiments may be implemented as a software program including instructions stored in a computer-readable storage medium.

A computer may refer to a device configured to retrieve an instruction stored in the computer-readable storage medium and to operate, in response to the retrieved instruction, and may include the ultrasound apparatus 100 according to embodiments.

The computer-readable storage medium may be provided in the form of a non-transitory storage medium. In this regard, the term ‘non-transitory’ means that the storage medium does not include a signal and is tangible, and the term does not distinguish between data that is semi-permanently stored and data that is temporarily stored in the storage medium.

In addition, the ultrasound apparatus 100 or the method according to embodiments may be provided in the form of a computer program product. The computer program product may be traded, as a product, between a seller and a buyer.

The computer program product may include a computer-readable storage medium having stored thereon the software program. For example, the computer program product may include a product (e.g. a downloadable application) in the form of a software program electronically distributed by a manufacturer of the ultrasound apparatus 100 or through an electronic market (e.g., Google™, Play Store™, and App Store™). For such electronic distribution, at least a part of the software program may be stored on the storage medium or may be temporarily generated. In this case, the storage medium may be a storage medium of a server of the manufacturer, a server of the electronic market, or a relay server for temporarily storing the software program.

In a system consisting of a server and a terminal (e.g., the ultrasound apparatus), the computer program product may include a storage medium of the server or a storage medium of the terminal. Alternatively, in a case where a third device (e.g., a smartphone) that communicates with the server or the terminal is present, the computer program product may include a storage medium of the third device. Alternatively, the computer program product may include a software program that is transmitted from the server to the terminal or the third device or that is transmitted from the third device to the terminal.

In this case, one of the server, the terminal, and the third device may execute the computer program product, thereby performing the method according to embodiments. Alternatively, at least two of the server, the terminal, and the third device may execute the computer program product, thereby performing the method according to embodiments in a distributed manner.

For example, the server (e.g., a cloud server, an artificial intelligence (AI) server, or the like) may execute the computer program product stored in the server, and may control the terminal to perform the method according to embodiments, the terminal communicating with the server.

As another example, the third device may execute the computer program product, and may control the terminal to perform the method according to embodiments, the terminal communicating with the third device. In more detail, the third device may remotely control the ultrasound apparatus 100 to emit an ultrasound signal to an object, and to generate an image of an inner part of the object, based on information about an ultrasound signal reflected from the object.

As another example, the third device may execute the computer program product, and may directly perform the method according to embodiments, based on at least one value input from an auxiliary device (e.g., a probe of a medical apparatus). In more detail, the auxiliary device may emit an ultrasound signal to an object and may obtain an ultrasound signal reflected from the object. The third device may receive an input of signal information about the reflected ultrasound signal from the auxiliary device, and may generate an image of an inner part of the object, based on the input signal information.

In a case where the third device executes the computer program product, the third device may download the computer program product from the server, and may execute the downloaded computer program product. Alternatively, the third device may execute the computer program product that is pre-loaded therein, and may perform the method according to the embodiments.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by one of ordinary skill in the art that various changes in form and details may be made therein without departing from the spirit and scope as defined by the following claims.

What is claimed is:

1. A method, performed by an ultrasound apparatus, of providing annotation-related information, the method comprising:
displaying, on a display of the ultrasound apparatus, an ultrasound image obtained by using a probe;
receiving an input from a user requesting an annotation setup interface;
identifying a diagnostic field corresponding to the ultrasound image;
determining text items, based on the identified diagnostic field; and
displaying, on a touchscreen of the ultrasound apparatus, the annotation setup interface comprising the text items.

2. The method of claim 1, wherein
the displaying of the annotation setup interface comprises determining a layout of the annotation setup interface by using at least one of an examination history, an examination posture, and physiological characteristic information.

3. The method of claim 1, wherein
the displaying of the annotation setup interface comprises determining a layout of the annotation setup interface by using at least one of information about with which hand a user uses the probe and information about a pattern by which the user selects text items.

4. The method of claim 1, wherein the determining of the text items comprises determining the text items by using measurement result information related to the ultrasound image.

5. The method of claim 4, wherein the determining of the text items comprises:
extracting at least one measurement result related to the ultrasound image; and
generating at least one text item corresponding to the at least one measurement result, and
wherein the displaying of the annotation setup interface comprises displaying the annotation setup interface including the at least one text item.

6. The method of claim 1, wherein the determining of the text items comprises determining the text items according to at least one of a type of the probe, a function of the ultrasound apparatus used in the obtaining of the ultrasound image, and information about a location in which the ultrasound apparatus is installed.

7. The method of claim 1, wherein the displaying of the annotation setup interface comprises:
grouping the text items according to semantic correlations; and
arranging the grouped text items on respective rows on the annotation setup interface.

8. The method of claim 1, further comprising:
receiving an input selecting one or more text items from among the text items;
generating an annotation by using the selected one or more text items; and
displaying the generated annotation on the ultrasound image.

9. The method of claim 8, wherein the input of selecting the one or more text items comprises a drag input of connecting the one or more text items.

10. The method of claim 8, further comprising:
when an input of selecting a button for adjusting a position of the annotation displayed on the ultrasound image is received, displaying, on the touchscreen, the ultrasound image and the annotation displayed on the display;
receiving a touch input of changing the position of the annotation with respect to the ultrasound image via the touchscreen; and
moving the annotation on the ultrasound image displayed on the display, in response to the touch input received via the touchscreen.

11. The method of claim 1, wherein the displaying of the annotation setup interface comprises determining, by using at least one of text item selecting pattern information and incorrect-input pattern information, at least one of positions of the text items comprised in the annotation setup interface, sizes of the text items, colors of the text items, fonts of texts comprised in the text items, sizes of the texts, and colors of the texts.

12. An ultrasound apparatus comprising:
a display configured to display an ultrasound image obtained by using a probe;
an input interface configured to receive an input user requesting an annotation setup interface; and
a processor configured to determine text items, based on a diagnostic field corresponding to the ultrasound image, and display the annotation setup interface comprising the text items on a touchscreen forming a portion of the input interface.

13. The ultrasound apparatus of claim 12, wherein the processor is further configured to determine a layout of the annotation setup interface by using at least one of an examination history, an examination posture, and physiological characteristic information, information about with which hand a user uses the probe with, and information about a pattern by which the user selects text items.

14. The ultrasound apparatus of claim 12, wherein the processor is further configured to determine the text items by using measurement result information related to the ultrasound image.

15. The ultrasound apparatus of claim 12, wherein the processor is further configured to determine the text items according to at least one of a type of the probe, a function of the ultrasound apparatus used in the obtaining of the ultrasound image, and information about a location in which the ultrasound apparatus is installed.

16. The ultrasound apparatus of claim 12, wherein the processor is further configured to group the text items according to semantic correlations, and arrange the grouped text items on respective rows on the annotation setup interface.

17. The ultrasound apparatus of claim 12, wherein the processor is further configured to receive an input of selecting one or more text items from among the text items via the touchscreen, generate an annotation by using the selected one or more text items, and control the display to display the generated annotation on the ultrasound image.

18. The ultrasound apparatus of claim 17, wherein the processor is further configured to, when an input of selecting a button for adjusting a position of the annotation displayed on the ultrasound image is received,
display, on the touchscreen, the ultrasound image and the annotation displayed on the display,
receive a touch input of changing the position of the annotation with respect to the ultrasound image via the touchscreen, and
move the annotation on the ultrasound image displayed on the display, in response to the touch input.

19. The ultrasound apparatus of claim 12, wherein the processor is further configured to determine, by using at least one of text item selecting pattern information and incorrect-input pattern information, at least one of positions of the text items comprised in the annotation setup interface, sizes of the text items, colors of the text items, fonts of texts comprised in the text items, sizes of the texts, and colors of the texts.

20. A computer program product comprising a computer-readable storage medium having a computer-readable program stored therein, wherein the computer-readable program, when executed on an ultrasound apparatus, causes the ultrasound apparatus to:
display, on a display of the ultrasound apparatus, an ultrasound image obtained using a probe;
receive an input user requesting an annotation setup interface;
identify a diagnostic field corresponding to the ultrasound image;
determine text items, based on the identified diagnostic field; and
display, on a touchscreen, the annotation setup interface comprising the text items.
